(19) United States
(12) Patent Application Publication
(10) Pub. No.: US 2011/0283375 A1
(43) Pub. Date: Nov. 17, 2011

(54) Transgenic Prothrombin And Related Thrombin Precursors And Transgenics, Methods, Compositions, Uses And The Like Relating Thereto

(75) Inventor: William Hugold Velander, Blacksburg, VA (US)

(73) Assignee: Bellweather Farms, East Woodstock, CT (US)

(21) Appl. No.: 13/150,936

(22) Filed: Jun. 1, 2011

Related U.S. Application Data
(62) Division of application No. 10/049,849, filed on Jun. 27, 2002, now abandoned.
(60) Provisional application No. 60/149,936, filed on Aug. 19, 1999.

(51) Int. Cl. A01K 67/027

(52) U.S. Cl. 800/15; 800/14; 800/16; 800/17

(57) ABSTRACT

The invention provides, among other things, activatable prothrombin, compositions comprising prothrombin, transgenic organisms for making prothrombin, methods for making the transgenic organisms, methods for making prothrombin-comprising compositions and for further purifying prothrombin from the compositions. Illustrative embodiments of the invention particularly provide transgenic mammals that express an exogenous gene for prothrombin and excrete the prothrombin encoded by the gene into their milk. In a highly particular illustrative embodiment in this regard the invention provides transgenic female pigs that express prothrombin in their milk. In this regard, the invention relates particularly to female pigs having stably incorporated in their genomes a DNA comprising a region that encodes prothrombin operably linked to a mammary gland-specific promoter. Further in this regard the invention relates to the milk containing the prothrombin and to prothrombin-containing compositions derived from the milk. It also relates to novel prothrombins obtained by expression of prothrombin genes in transgenic organisms and to uses, therapeutic and otherwise, of the prothrombins.

This application claims benefit of U.S. Provisional application No. 60/149,936 of William Velander for Transgenic Prothrombin and Related Thrombin Precursors and Transgenics, Methods, Compositions, Uses and the like Relating Thereto filed 19 Aug. 1999, which is incorporated herein by reference in its entirety.

### FIELD OF THE INVENTION

The invention provides, among other things, activatable prothrombin, compositions comprising prothrombin, transgenic organisms for making prothrombin, methods for making the transgenic organisms, methods for making prothrombin-comprising compositions and for further purifying prothrombin from the compositions. Illustrative embodiments of the invention particularly provide transgenic mammals that express an exogenous gene for prothrombin and excrete the prothrombin encoded by the gene into their milk. In a highly particular illustrative embodiment in this regard the invention provides transgenic female pigs that express prothrombin in their milk. In this regard, the invention relates particularly to female pigs having stably incorporated in their genomes a non-endogenous DNA comprising a region that encodes prothrombin operably linked to a mammary gland-specific promoter. Further in this regard the invention relates to the milk containing the prothrombin and to prothrombin-containing compositions derived from the milk. It further relates in these and other respects to novel prothrombin and prothrombin-related polypeptides obtained by expression of prothrombin and prothrombin-related genes in transgenic organisms. And it also relates to, among other things, uses of the prothrombin and prothrombin-related polypeptides, including therapeutic and other uses.

### BACKGROUND

Prothrombin (also called Factor II and F2), is a crucial blood-borne component of the coagulation cascade. At sites of injury prothrombin is converted to thrombin, which catalyzes the formation and cross-linking of fibrin clots by cleaving fibrinogen into self-polymerizing fibrin monomers and activating the cross-linking activity of Factor XIII. Prothrombin thus is the physiological storage repository for procoagulant clotting potential that can be converted immediately to thrombotic activity at sites of injury to staunch bleeding and simulate immune and healing responses. (For a review see PROTHROMBIN AND OTHER VITAMIN K PROTEINS Vols I and II, Seegers and Walz, Eds., CRC Press, Boca Raton, Fla. (1986) which is incorporated herein by reference in its entirety, particularly parts pertinent to prothrombin structure, modification, activity, production, purification, physiological activity, functions and effects, and uses including clinical and non-clinical uses.)

Human prothrombin is fairly typical of mammalian prothrombins. It is a single chain protein. It contains a pro peptide, a gla domain, two kringle regions, an A chain and a serine protease domain. It also contains two sites for cleavage by factor Xa.

Prothrombin is activated to thrombin by a series of proteolytic cleavages. The circulating single chain zymogen is activated by Factor Xa complex, which cleaves prothrombin at two sites. Cleavage at the first site liberates a fragment containing the gla domain and the two kringle regions. This N-terminal fragment, referred to as Fragment 1.2, contains the moieties responsible for calcium bridge formation and for the interaction of prothrombin with Factor V. The C-terminal fragment, referred to as Prethrombin 2, is the immediate, thrombotically inactive precursor of thrombin. Prethrombin 2 is activated by the second cleavage by Factor Xa complex. The second cleavage splits Prethrombin 2 into two chains linked by disulfide bonds. The disulfide-linked two-chain molecule is active thrombin. (See, for instance, pages 514-516 in TEXTBOOK OF HEMATOLOGY, 2nd Edition, Shirlyn B. McKenzie, William & Wilkins, Baltimore (1996)) which is herein incorporated herein by reference in parts pertinent to thrombin and prothrombin.)

Prothrombin and thrombin exhibit a variety of post-translational modifications. Many of the modifications regulate activities of the proteins and are important to their physiological functions. Important modifications include proteolytic processing, as described above, glycosylation and glutamic acid γ-carboxylation as discussed further below.

Human and bovine prothrombin, for instance, are γ-carboxylated at glutamic acid residues 7, 8, 15, 17, 20, 21, 26, 27, 30 and 33 by a series of vitamin K-dependent enzyme reactions. Mouse and rat have the same sites for glutamic acid carboxylation and likely exhibit the same pattern of γ-carboxylation. (See for instance Degen, Seminars in Thrombosis and Hemostasis 18(2): 230-242 (1992) which is incorporated herein by reference in its entirety, particularly as to the foregoing in parts pertinent to γ-carboxylation of prothrombins.) Gamma-carboxylation of some of the residues is required for calcium-dependent membrane binding and thus plays an important role in localizing prothrombin at sites of injury. Gamma carboxylation of other glutamic acid residues modulates interaction and complex formation of prothrombin with other vitamin K-dependent coagulation factors. Physiologically, particularly in humans, it appears that complete-carboxylation is required for activation and conversation of prothrombin to thrombin. Notably, the extent of γ-carboxylation of prothrombin varies markedly from one preparation to another, even for preparations made in the same system according to the same protocol.

Although physiological activation of prothrombin requires complete γ-carboxylation, it is not necessary for thrombin activity. In fact, all the sites for prothrombin γ-carboxylation occur in a relatively small region, called the “gla domain,” near the carboxyl terminus. Proteolytic cleavage during activation separates the entire gla domain from the regions of prothrombin that form thrombin and, as a result, there are no carboxylation sites in thrombin. Since active thrombin does not require γ-carboxylation it can be derived by chemical and other cleavage methods from prothrombin with or without γ-carboxylation.

Prothrombin also is glycosylated. Human prothrombin contains three sites for N-linked glycosylation: Asn-79, Asn-101 and Asn-378. An additional site, Asn-Leu-Ser at Asn-165, matches the consensus Asn-X-Ser/Thr sequence of N-linked glycosylation; but, does not appear to be glycosylated in human prothrombin. Bovine prothrombin is similarly glycosylated at three sites. Two of the bovine sites, Asn-101 and Asn-378, are the same as human prothrombin; but, the third site in bovine prothrombin is Asn-77 rather than Asn-79. Mouse prothrombin has five sites for N-linked glycosylation: Asn-79, Asn-101, Asn-165, Asn-378 and Asn-518. Rat prothrombin also has five sites. Four are identical to mouse; but, one is different: Asn 79 rather than 77. The mouse and rat sites at Asn-165, analogous to the human site, probably are not glycosylated. The extent and types of glycosylation observed at these sites varies considerably in all organisms in which it has been studied. (For instance see Degen, Seminars in Thrombosis and Hemostasis 18(2): 230-242 (1992) which is incorporated herein by reference in its entirety, as to the foregoing particularly with regard to glycosylation of prothrombins.)

Glycosylation plays an important role in activity and physiological function and effects of prothrombin Generally, glycosylation can affect enzymatic activity, substrate preferences, binding to cofactors and other moieties, complex formation, thermal stability, resistance to proteases and physiological persistence among other things. (For instance see PROTHROMBIN AND OTHER VITAMIN K PROTEINS Vols I and II, Seegers and Walz, Eds., CRC Press, Boca Raton, Fla. (1986) which is incorporated herein by reference in its entirety, as to the foregoing particularly in parts pertinent to glycosylation of prothrombin, especially in this regard Vol. 1, Chapter 8, Kobata and Mizuochi, Current Status of Carbohydrate Constituents and Prospects, 81-94.)

Prothrombin and thrombin have a wide variety of clinical and non-clinical applications. The most important of these, at present, relate to the thrombotic activity of thrombin and its application in veterinary and human clinical circumstances. Among clinically important applications thrombin is used to promote hemostasis, to improve anastomoses, to control hemorrhage, to achieve good hemostasis on bone defects, to seal vascular prostheses, to seal lesions and stumps, to treat pleurodesis, to close fistulas. to seal membranes, in procedures to extract stones and to prevent or reduce perioperative bleeding, to mention just a few. (See PROTHROMBIN AND OTHER VITAMIN K PROTEINS Vols I and II, Seegers and Walz, Eds., CRC Press, Boca Raton, Fla. (1986) which is incorporated herein by reference in its entirety, as to the foregoing particularly in parts pertinent to uses of prothrombin and thrombin, especially in this regard Vol. II. Chapter 7, Deutsch, The Clinical Use of Thrombin, 92-103.)

In particular, as to uses relating to hemostasis per se, thrombin has been used in a wide variety of applications including but not limited to promoting hemostasis in animals and humans at, to name a few, lacerations and other wounds, sites of organ rupture, sites of bleeding during surgery, bum sites, sites of traumatic injury, surgical sites such as partial resections, including partial brain resections, bleeding biopsies, sites of tumor extirpation, including tumors from parenchymatous organs such as liver, spleen, pancreas, kidney, brain and prostatic gland among others, sites of donations of skin grafts, sites of skin grafts, after extraction of teeth, nose bleeding, sinus bleeding, bleeding in or near bones, gastrointestinal bleeding, and conjunctival wounds, to name but a few. As to anastomoses, thrombin has been used to, among other things, tighten classically sutured anastomoses, to reduce the number of sutures in, for example, anastomoses of intestines, small vessels, maxillo-facial vessels and extracranial anastomoses, to prevent kinking of arterial grafts, and to promote the combination of nerve endings, to name but a few.

Among particularly important applications in this regard is the use of thrombin to promote hemostasis in surgery and of wounds associated with trauma, particularly wounds in civilian and military personnel that result from warfare.

Both prothrombin and thrombin have been isolated from natural sources. Commercial thrombin preparations have been made from pooled human blood and from pooled animal blood. Unfortunately, preparations from pooled starting material, in which it is an incidental component, generally varies enormously in composition and quality. Preparations derived from human sources, moreover, always pose a great danger of contamination with pathogens and other deleterious substances. Preparations from animal sources also pose a contamination threat and furthermore may induce adverse immune reactions. (See for instance PROTHROMBIN AND OTHER VITAMIN K PROTEINS Vols I and II, Seegers and Walz, Eds., CRC Press, Boca Raton, Fla. (1986) which is incorporated herein by reference in its entirety, particularly in parts pertinent to clinical and other uses of prothrombin and thrombin, especially in this regard Vol. II, Chapter 10, Murano, Commercial Preparations of Vitanzin K-Dependent Factors and Their Use In Therapy, 131-142.)

Production using recombinant DNA technology to express cloned genes that encode prothrombin and related polypeptides in cells in culture has been viewed as the best alternative to isolating prothrombin from natural sources, such as animal or human plasma. Potentially, cell expression systems would eliminate many of the risks of potential contamination associated with natural sources, such as the risk of contamination with blood-borne viruses. Moreover, cell expression methods, theoretically if not practically, could provide a sufficient supply of well characterized and reliable product to meet most projected needs.

Nevertheless, the complex post-translational modifications of prothrombin, such as those discussed above that are necessary for physiological activities, pose a difficult obstacle to the production of active prothrombin or thrombin polypeptides in cells using cloned genes. Moreover, attempts to culture genetically altered cells to produce prothrombin polypeptides have produced uneconomically low yields and, generally, preparations of low specific activity. Apparently, the post-translational modification systems in the host cells could not keep pace with production of exogenously encoded protein, reducing specific activity. In addition, the capacity of engineered cells to produce prothrombin thus has been limited. Thus, cell culture production methods have not provided hoped for advantages for producing prothrombin reliably and economically.

Another way to produce these prothrombin is in transgenic organisms. However, it is most likely that only mammals will be able to carry out the post-translational modifications necessary for physiological function of prothrombin. Furthermore, inappropriately active thrombin would pose a deadly risk to a transgenic animal. Whether engendered by expression of thrombin or by activation of expressed prothrombin, inappropriate thrombin activation likely would lead to massive vascular clotting and death. Even compartmentalization seems unlikely to provide adequate protection against this possibility, since other carefully compartmentalized polypeptides, such as proteins specifically expressed in milk, nevertheless are detected in the blood of the transgenic animals. Apparently the circulatory system always has some communication with other bodily compartments, such as mammary glands, through which at least small amounts of proteins that otherwise are tightly compartmentalized nonetheless can enter the blood stream. It has not been possible, as yet, to produce prothrombin, thrombin or related polypeptides from a controlled source in a highly active form with a good yield and there exists a need for better methods to produce prothrombin and prothrombin-related polypeptides.

### DESCRIPTION OF THE INVENTION

It is therefore an object of the present invention to provide a transgenic organism that produces prothrombin and prothrombin-related polypeptides, particularly prothrombin and prothrombin-related polypeptides that are thrombotically inactive but which can be activated to provide thrombotic activity.

The invention is directed to a transgenic organism comprising an introduced genetic construct that engenders production of a prothrombin or a prothrombin-related polypeptide.

The invention is also directed to a transgenic organism as above where the construct engenders production of a prothrombin or prothrombin-related polypeptide in specific cells.

The invention is also directed to a transgenic organism as above where the prothrombin or prothrombin-related polypeptide accumulates in a specific tissue or bodily compartment.

The invention is also directed to a transgenic organism as above where the prothrombin or prothrombin-related polypeptide accumulates in a bodily fluid.

The invention is also directed to a transgenic organism as above where the organism is a non-human mammal.

The invention is also directed to a transgenic organism as above where the mammal is a mouse, rat, hamster, rabbit, pig, sheep, goat, cow or horse.

The invention is also directed to a transgenic organism as above where the organism is a mouse, pig, sheep, goat or cow.

The invention is also directed to a transgenic organism as above where the organism is a pig.

The invention is also directed to a transgenic organism as above where the prothrombin or prothrombin-related polypeptide accumulates in the milk of females.

The invention is also directed to a transgenic organism as above where the prothrombin or prothrombin-related polypeptide produced in the organism when isolated and purified has a specific activity 50% to 75% of that of purified human prothrombin.

The invention is also directed to a transgenic organism as above where the specific activity is 70% to 85% of that of purified human prothrombin.

The invention is also directed to a transgenic organism as above where the specific activity is 80% to 95% of that of purified human prothrombin.

The invention is also directed to a transgenic organism as above where the specific activity is 85% to 98% of that of purified human prothrombin.

The invention is also directed to a transgenic organism as above where the specific activity is 90% to 105% of that of purified human prothrombin.

The invention is also directed to a transgenic organism as above where the specific activity is 75% to 125% of that of purified human prothrombin.

The invention is also directed to a transgenic organism as above where the specific activity is 50% to 110% of that of purified human prothrombin.

The invention is also directed to a transgenic organism as above where the specific activity is more than that of purified human prothrombin.

The invention is also directed to a transgenic organism as above where activity is determined by APTT assay.

The invention is also directed to a transgenic organism as above where activity is determined by a calcium-dependent membrane-binding dependent assay.

The invention is also directed to a transgenic organism as above where activity is determined by an assay that does not require membrane binding.

The invention is also directed to a transgenic organism as above where the prothrombin or prothrombin related polypeptide comprises a region having an amino acid sequence 80% to 100% identical to that of a mammalian thrombin.

The invention is also directed to a transgenic organism as above where the prothrombin or prothrombin related polypeptide comprises a region having an amino acid sequence 90% to 100% identical to that of a mammalian thrombin.

The invention is also directed to a transgenic organism as above where the prothrombin or prothrombin related polypeptide comprises a region having an amino acid sequence 95% to 100% identical to that of a mammalian thrombin.

The invention is also directed to a transgenic organism as above where the mammalian thrombin is human thrombin.

The invention is also directed to a transgenic organism as above where the prothrombin or prothrombin-related polypeptide comprises a region having the amino acid sequence of human thrombin.

The invention is also directed to a transgenic organism as above where the prothrombin or prothrombin related polypeptide comprises a region having an amino acid sequence 80% to 100% identical to that of a mammalian prothrombin.

The invention is also directed to a transgenic organism as above where the prothrombin or prothrombin related polypeptide comprises a region having an amino acid sequence 90% to 100% identical to that of a mammalian prothrombin.

The invention is also directed to a transgenic organism as above where the prothrombin or prothrombin related polypeptide comprises a region having an amino acid sequence 95% to 100% identical to that of a mammalian prothrombin.

The invention is also directed to a transgenic organism as above where the mammalian thrombin is human prothrombin.

The invention is also directed to a transgenic organism as above where the prothrombin or prothrombin-related polypeptide comprises a region having the amino acid sequence of human prothrombin.

The invention is also directed to a transgenic organism as above where the prothrombin or prothrombin-related polypeptide is human prothrombin.

The invention is also directed to a transgenic organism as above where the introduced genetic construct comprises a promoter operatively linked to the region encoding prothrombin or a prothrombin-related polypeptide, wherein further the promoter is selected from the group consisting of the promoters of whey acidic protein genes, casein genes, lactalbumin genes and beta lactoglobulin genes.

The invention is also directed to a transgenic organism as above where the promoter is a whey acidic protein promoter or a beta lactoglobulin promoter.

The invention is also directed to transgenic organisms as above where the promoter is a whey acidic protein promoter.

The invention is also directed to a transgenic organism as above where the promoter is the mouse whey acidic protein promoter, the rat whey acidic protein promoter or the pig whey acidic protein promoter.

The invention is also directed to a transgenic organism as above where the promoter is a long whey acidic protein promoter.

The invention is also directed to a transgenic organism as above where the promoter is the mouse long whey acidic protein promoter.

The invention is also directed to a composition comprising prothrombin or a prothrombin-related polypeptide produced in a transgenic organism as above described.

The invention is also directed to a composition as above where the prothrombin or prothrombin-related polypeptide produced is from milk of a non-human transgenic female mammal.

The invention is also directed to a composition as above where the composition is milk of the transgenic mammal.

The invention is also directed to a composition as above where the composition is derived from milk of the transgenic mammal.

The invention is also directed to a prothrombin or prothrombin-related polypeptide isolated from a transgenic organism as above described.

The invention is also directed to a prothrombin or prothrombin-related polypeptide isolated from a transgenic organism as above that differs in its post-translational modification from that of naturally occurring human prothrombin.

The invention is also directed to a human prothrombin polypeptide isolated from a transgenic organism as above that differs in post-translational modification from human prothrombin isolated from natural sources but that has the same thrombotic activity.

The invention is also directed to a human prothrombin polypeptide isolated from a transgenic organism as above that differs in post-translational modification from human prothrombin isolated from natural sources but that has the same physiological activities.

The invention is also directed to a prothrombin or prothrombin-related polypeptide isolated from a transgenic organism as above that differs in its glycosylation from that of human prothrombin.

The invention is also directed to a human prothrombin polypeptide isolated from a transgenic organism as above that differs in glycosylation from human prothrombin isolated from natural sources but has the same thrombotic activities.

The invention is also directed to a human prothrombin polypeptide isolated from a transgenic organism as above that differs in glycosylation from human prothrombin isolated from natural sources but has the same physiological activities.

The invention is also directed to a prothrombin or prothrombin-related polypeptide isolated from a transgenic organism as above that differs in its γ-carboxylation from that of human prothrombin isolated from natural sources.

The invention is also directed to a prothrombin or prothrombin-related polypeptide isolated from a transgenic organism as above that differs in its γ-carboxylation from that of human prothrombin isolated from natural sources but has the same thrombotic activity.

The invention is also directed to a composition for treating a wound comprising a thrombin derived from a prothrombin or prothrombin-related polypeptide as described above. The invention is also directed to a method for treating a wound in a patient comprising a step of contacting the wound with prothrombin or a prothrombin-related polypeptide as above described.

The invention is also directed to a method for treating a wound in a patient comprising a step of contacting the wound with thrombin or a thrombin-related polypeptide derived from prothrombin or a prothrombin-related polypeptide as above described.

The invention is also directed to a method for producing prothrombin or a prothrombin-related polypeptide comprising the step of producing the prothrombin or prothrombin-related polypeptide in a transgenic organism as above described.

In accomplishing the foregoing objects, there has been provided, in accordance with one aspect of the present invention, a transgenic mammal containing an exogenous DNA sequence stably integrated in its genome, wherein the exogenous DNA sequence comprises a promoter operably linked to a DNA sequence encoding a polypeptide that, when activated, has thrombin activity and a signal peptide, wherein the promoter is specifically active in mammary gland cells, and the signal peptide is effective in directing the secretion of the polypeptide from the cells into the milk of the transgenic mammal.

In a preferred embodiment, the promoter is a whey acidic protein promoter.

In accordance with another aspect of the present invention, there has been provided a process for the production of a polypeptide that can be activated to provide thrombin activity, comprising the steps of (A) providing a transgenic mammal characterized by an exogenous DNA sequence stably integrated in its genome, wherein the exogenous DNA sequence comprises a promoter operably linked to a DNA sequence encoding a polypeptide that when activated has thrombin activity and a signal peptide, the promoter being specifically active in mammary cells and the signal peptide being effective in directing the secretion of the polypeptide into the milk of the transgenic mammal; (B) producing milk from the transgenic mammal; (C) collecting the milk; and (D) isolating the polypeptide from the milk. In one preferred embodiment, the transgenic mammal is mouse, rabbit, pig, sheep or goat. In an especially preferred embodiment the transgenic mammal is pig.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

Notwithstanding the apparent disadvantages of currently available methods for obtaining prothrombins and prothrombin-related polypeptides that provide thrombotic activities, such as clinically useful procoagulant activities, the present invention provides, among other things, transgenic organisms that express prothrombins and related polypeptides that provide thrombotic activities. Prothrombins and related polypeptides in preferred embodiments in this regard are polypeptides and proteins that are sources of thrombin and thrombin-related polypeptides, particularly thrombin and thrombin-related polypeptides having activities of thrombins, particularly physiological activities and activities useful in clinical applications. In some preferred embodiments of certain aspects of the invention , preferred prothrombin and related polypeptides are inactive and can be activated as to a particular activity or activities, such as an enzymatic, binding or other activity. In other preferred embodiments of the invention in certain aspects of the invention in this regard the polypeptides are active and need not be activated. In certain further aspects of the invention preferred prothrombin and related polypeptides are those that have or can be activated to have one or more activities of thrombin, particularly thrombotic activity, as further described herein. Thus, in one aspect the invention provides in certain preferred embodiments polypeptides that comprise a region or regions that provide for thrombotic activity. In certain embodiments preferred in this regard in certain aspects of the invention the polypeptides are polypeptides of amino acid sequence comprising a region or regions of amino acid sequence providing thrombotic activity or activities. Furthermore, as described below, the present invention also provides, among other things, methods for obtaining the prothrombins and related polypeptides from the transgenic organisms, compositions comprising transgenically produced prothrombins and related polypeptides, and uses thereof, to name a few, as described in greater detail below.

### METHODS FOR MAKING TRANSGENIC ORGANISMS

Transgenic organisms that express prothrombin and related polypeptides may be produced in accordance with the invention as described herein using a wide variety of well-known techniques, such as those described in GENETIC ENGINEERING OF ANIMALS, Ed. A. Puhler, VCH Publishers, New York (1993) and in more detail in Volume 18 in Methods in Molecular Biology: TRANSGENESIS TECHNIQUES, Eds. D. Murphy and D. A. Carter, Humana Press, Totowa, N.J. (1993) both of which are incorporated herein by reference in their entireties, particularly as to the foregoing in parts pertinent to methods for making transgenic organisms that express prothrombin and related polypeptides, especially in milk. (See also for instance Lubon et al., Transfusion Medicine Reviews X(2): 131-141 (1996) which is incorporated herein by reference in its entirety, particularly as to the foregoing in parts pertinent to methods for making transgenic organisms.

In particular, transgenic mammals, such as mice and pigs, that express prothrombin or other prothrombin-related polypeptides in accordance with certain preferred embodiments of the invention, can be produced using methods described in among others MANIPULATING THE MOUSE EMBRYO, Hogan et al., Cold Spring Harbor Press (1986); Krimpenfort et al., Bio/Technologv 9: 844 et seq. (1991); Palmiter et al., Cell 42: 343 et seq. (1985); GENETIC MANIPULATION OF THE EARLY MAMMALIAN EMBRYO, Kraemer et al., Cold Spring Harbor Press, Cold Spring Harbor, N.Y. (1985); Hammer et al., Nature 315: 680 et seq. (1985); U.S. Pat. No. 4,873,191 of Wagner et al. for Genetic Transformation of Zygotes, and U.S. Pat. No. 5,175,384 of Krimpenfort et al. for Transgenic Mice Depleted in Mature T-Cells and Methods for Making Transgenic Mice, each of which is incorporated herein by reference in its entirety. particularly as to the foregoing in parts pertinent to producing transgenic mammals by introducing DNA or DNA:RNA constructs for polypeptide expression into cells or embryos. (Regarding DNA:RNA constructs and their use see for instance U.S. Pat. No. 5,565,350 of Kmiec for Compounds and Methods for Site Directed Mutations in Eukarvoric Cells and U.S. Pat. No. 5,756,325 of Kmiec also for Compounds and Methods for Site Directed Mutations in Eukaryotic Cells each of which is incorporated by reference herein in its entirety particularly as to the foregoing in parts pertinent to targeted genetic manipulations useful to produce transgenic organisms that express prothrombin or a prothrombin-related polypeptide.

For example, transgenic organisms of the present invention can be produced by introducing into eggs or developing embryos one or more genetic constructs that engender expression of prothrombin or related polypeptides as described herein. In certain preferred embodiments of the invention in this regard, DNAs that comprise cis-acting transcription controls for expressing prothrombin operably linked to a region encoding prothrombin are highly preferred. RNA-DNA hybrids are similarly preferred in some embodiments in this regard. Also useful in this regard are constructs that engender non-natural expression of genes for prothrombin or prothrombin related polypeptides. Constructs that comprise operable signal sequences that effectuate transport of the prothrombin polypeptides into a targeted compartment of an organism, such as a tissue or fluid, are further preferred in certain embodiments in this regard. Also especially preferred in this regard are constructs that are stably incorporated in the genome of germ line cells of the mature organism and inherited in normal, Mendelian fashion by reproduction thereof. One or more DNA or RNA:DNA hybrids or the like may be used alone or together to make transgenic organisms useful in the invention as described further below.

Standard techniques, as well as unusual and new techniques for making transgenic organisms generally can be used to make transgenic organisms that express prothrombin and related polypeptides in accordance with the invention. Useful techniques in this regard include those that introduce genetic constructs by injection, infection, transfection, such as calcium phosphate transfection, using cation reagents, using sperm or sperm heads or the like, lipofection, liposome fusion, electroporation, and ballistic bombardment, to name just a few known techniques. Useful techniques include those that involve homologous recombination, such as those that can be employed to achieve targeted integration, and those that do not, such as those disclosed below.

Constructs can be introduced using these and other methods into pluripotent cells, totipotent cells, germ line cells, eggs, embryos at the one cell stage, and embryos at several cell stages, among others, to make transgenic organisms of the invention. In these regards, among others, they may be introduced by such methods in pronuclei, nuclei, cytoplasm or other cell compartments or into extracellular compartments of multicellular systems to make transgenic organisms of the invention.

In a preferred method, developing embryos can be infected with retroviral vectors and transgenic animals can be formed from the infected embryos. In a particularly preferred method DNAs in accordance with the invention are injected into embryos, preferably at the single-cell stage. In some particularly preferred embodiments in this regard, DNA is injected in the pronucleus of a one-cell embryo. In other preferred embodiments in this regard, DNA is injected into the cytoplasm of a one cell embryo. In yet another particularly preferred embodiment in this regard, DNA is injected into an early stage, several cell embryo In these regards, in like manner, in yet other preferred embodiments a DNA-RNA hybrid is injected into an embryo, particularly single-cell embryos, into the pronucleus or the cytoplasm, or into an early stage embryo.

### CONSTRUCTS FOR TRANSGENIC EXPRESSION

Certain aspects of the invention relate to the introduction into organisms of genetic constructs that engender expression of prothrombin and related polypeptides. Such constructs are referred to as, among other things, transgenic elements, transgenes, introduced genes, introduced genetic elements, exogenous genes, exogenous genetic elements, exogenously derived genetic elements and the like. As noted in greater detail elsewhere herein such elements may encode the expressed polypeptide, they may alter the control of expression of a polypeptide in the host, they may alter the amino acid sequence of the polypeptide in the host or a combination of these, among others. They have as a general property as to the present invention that they alter the host organism and engender expression that does not otherwise occur therein of prothrombin and related polypeptides as disclosed in greater detail elsewhere herein.

Among the genetic constructs and the like that are useful in the invention in this regard are polynucleotide constructs that provide a DNA sequence encoding prothrombin or other thrombin-related polypeptide of the invention operably linked to cis-acting signals necessary for expression in a transgenic organism and, in certain preferred embodiments, for transport of a translation product encoded by the construct into a particular compartment of the organism. Among preferred polynucleotides for constructs in preferred embodiments of the invention are DNA or RNA:DNA hybrids. Among particularly preferred embodiments in this regard are DNA polynucleotides.

The constructs may be a single polynucleotide or several polynucleotides when introduced into a cell or embryo or the like to form a transgenic animal in accordance with the invention. Particularly preferred are single chain, double-stranded DNA polynucleotides in this regard. Also preferred are DNA-RNA hybrid polynucleotides. When more than one polynucleotide is used in this regard, they generally combine with one another and/ or with endogenous genetic elements of the host organism, as a result of in vitro or in vivo processes, to form a construct that then engenders transgenic expression of the prothrombin or related polypeptides in the host organism.

In certain particularly preferred embodiments of the invention, preferred constructs provide a polynucleotide sequence encoding prothrombin or other prothrombin-related polypeptide of the invention, operably linked to the cis-acting signals necessary for expression in mammary gland cells and for secretion into milk of a non-human female transgenic mammal. Particularly highly preferred in this regard are cis-acting signals that provide efficient expression in mammary glands and secretion into milk of highly active prothrombin with little or no expression elsewhere in the organism, as described in greater detail elsewhere herein. DNA and RNA:DNA hybrids are particularly preferred polynucleotides in this regard. DNA is especially preferred.

### PROTHROMBIN AND PROTHROMBIN-RELATED POLYPEPTIDES

In a particular aspect the invention provides human prothrombin and prothrombin-related polypeptides that can be activated to provide thrombotic activity. Particularly preferred embodiments in this regard provide prothrombin having the amino acid sequence of naturally occurring human prothrombin. The invention further provides prothrombin-related polypeptides that provide thrombotic activity. Preferred embodiments in this regard provide prothrombin-related polypeptides that are thrombotically inactive until they are activated to provide thrombotic activity. Further preferred embodiments in this regard provide prothrombin-related polypeptides that comprise thrombin having the amino acid sequence of naturally occurring human thrombin. Especially preferred embodiments in this regard provide prothrombin-related polypeptides that upon activation release human thrombin. In another aspect, among such preferred embodiments are prothrombin-related polypeptides more resistant to protease degradation in mammary epithelial cells and milk than naturally occurring human prothrombin.

Preferred embodiments of the invention in this regard in particular provide prothrombin and prothrombin-related polypeptides that are homologous to human prothrombin and can be activated to provide thrombotically active thrombin or thrombin-related polypeptides. Particularly preferred polypeptides in this regard comprise a region that is 70% or more, especially 80% or more, more especially 90% or more, yet more especially 95% or more, particularly 97% or more, more particularly 98% or more, yet more particularly 99% or more identical in amino acid sequence to the corresponding amino acid sequence of naturally occurring human prothrombin.

Identity in this regard can be determined using a variety of well known and readily available amino acid sequence analysis software. Preferred software includes those that implement the Smith-Waterman algorithm, considered a satisfactory solution to the problem of searching and aligning sequences. Other algorithms also may be employed, particularly where speed is an important consideration. Commonly employed programs for alignment and homology searching DNAs, RNAs and polypeptides that can be used in this regard include FASTA, TFASTA, BLASTN, BLASTP, BLASTX, TBLASTN, PROSRCH, BLAZE and MPSRCH, the latter being an implementation of the Smith-Waterman algorithm for execution on massively parallel processors made by MasPar.

The BLASTN, BLASTX and BLASTP programs are among preferred programs for homology determinations, the former for polynucleotide sequence comparisons and the latter two for polypeptide sequence comparisons—BLASTX for comparison of the polypeptide sequences from all three reading frames of polynucleotide sequence and BLASTP for a single polypeptide sequence. BLAST provides several user definable parameters that are set before implementing a comparison, including the following. (1) A value is set for E to establish the number of High Scoring Segment Pairs expected by chance. (2) A value is set for S to establish the cut-off score for reporting a High Scoring Segment Pair, i.e., for listing a segment pair as a significant match. Usually S is calculated from E. The values of E and S calculated for a given search string will be different on different databases. Accordingly, the values chosen for E and for the S cut off often are different for different databases. To normalize between different databases a parameter called Z is used. While the use of sophisticated techniques for setting E and S are entirely consistent with the present invention, a presently preferred method for determining similarity and homology of sequences using BLAST is to set S to the default value (10) and to calculate E from the default value of S using the default setting in the BLAST program being employed.

Identity and homology determining methods are discussed in, for instance, GUIDE TO HUMAN GENOME COMPUTING, Ed. Martin J. Bishop, Academic Press, Harcourt Brace & Company Publishers, New York (1994), which is incorporated herein by reference in its entirety with regard to the foregoing particularly in parts pertinent determining identity and or homology of amino acid or polynucleotide sequences, especially Chapter 7. The BLAST programs are described in Altschul et al., “Basic Local Alignment Research Tool”, J Mol Biol 215: 403-410 (1990), which is incorporated by reference herein in its entirety. Additional information concerning sequence analysis and homology and identity determinations are provided in, among many other references well known and readily available to those skilled in the art: NUCLEIC ACID AND PROTEIN

SEQUENCE ANALYSIS: A PRACTICAL APPROACH, Eds. M. J. Bishop and C. J. Rawings, IRL Press, Oxford, UK (1987); PROTEIN STRUCTURE: A PRACTICAL APPROACH, Ed., T. E. Creighton, IRL Press, Oxford, UK (1989); Doolittle, R. F.: “Searching through sequence databases” Met Enz 183: 99-110 (1990); Meyers and Miller: “Optimal alignments in linear space” Comput Applica in Biosci 4: 11-17 (1988);

Needleman and Wunsch: “A general method applicable to the search for similarities in amino acid sequence of two proteins” J Mol Biol 48: 443-453 (1970) and Smith and Waterman “Identification of common molecular subsequences” J Mol Biol 147: 1950 et seq. (1981), each of which is incorporated herein by reference in its entirety with reference to the foregoing particularly in parts pertinent to sequence comparison and identity and homology determinations.

Among preferred embodiments in this regard are those that provide prothrombin and or prothrombin-related polypeptides that provide, when activated, high thrombotic activity, especially high thrombotic activity as determined by activated partial thromboplastin time assay, particularly using a standard human plasma preparation for comparison. The measure of prothrombin activity in common use is the thrombin activity of prothrombin following prothrombin activation to thrombin. Assays relating to thrombiniprothrombin activity are widely known in the art and are described in greater detail elsewhere herein.

Especially preferred in this regard are prothrombin and prothrombin-related polypeptides comprising a region having an amino acid sequence with an aforementioned degree of identity to the amino acid sequence of naturally occurring human prothrombin. Among the most especially preferred in this regard are those comprising a region having the amino acid sequence of human thrombin. Especially preferred in this regard are prothrombins having the amino acid sequence of naturally occurring human prothrombin.

In this regard especially preferred embodiments are those that have 50% or more of the thrombotic activity of a standard reference preparation of thrombotically active human plasma-derived prothrombin, particularly as measured by activated partial thromboplastin time assay. Particularly highly preferred embodiments in this regard have 65% or more of the activity of the aforementioned reference preparation, yet more highly preferred embodiments in this regard have 75% or more of the activity of the reference, preferably 85% or more, yet more preferably 90% or more, still yet more preferably 95% or more.

Other particularly preferred embodiments in this regard have 50% to 150% of the activity of the aforementioned reference preparation. Particularly highly preferred embodiments in this regard have 60% to 125% of the activity of the reference preparation. Yet more highly preferred embodiments have 75% to 110% of the activity of the reference preparation. Still more highly preferred embodiments have 85% to 125% the activity of the reference. Still more highly preferred embodiments have 90% to 110% of the activity of the reference.

Among particularly preferred embodiments in this regard are those that provide prothrombin and or prothrombin-related polypeptides that have high specific thrombotic activity, particularly high specific activity as determined by activated partial thromboplastin time assay especially using a standard human plasma preparation for comparison. Especially preferred in this regard are prothrombin and prothrombin-related polypeptides comprising a region having an amino acid sequence with an aforementioned degree of identity to the amino acid sequence of naturally occurring human prothrombin. Among the most especially preferred in this regard are those comprising a region having the amino acid sequence of human thrombin. Especially preferred in this regard are prothrombins having the amino acid sequence of naturally occurring human prothrombin.

In this regard especially preferred embodiments are those that have 50% or more of the thrombotic activity of a standard reference preparation of thrombotically active human plasma-derived prothrombin, particularly as measured by activated partial thromboplastin time assay. Particularly highly preferred embodiments in this regard have 65% or more of the activity of the aforementioned reference preparation, yet more highly preferred embodiments in this regard have 75% or more of the activity of the reference, preferably 85% or more, yet more preferably 90% or more, still yet more preferably 95% or more of the activity of the reference preparation.

Other particularly preferred embodiments in this regard have 50% to 150% of the activity of the aforementioned reference preparation, particularly highly preferred embodiments in this regard have 60% to 125% of the activity of the reference preparation, yet more highly preferred embodiments have 75% to 110% of the activity of the reference preparation, still more highly preferred embodiments have 85% to 125% the activity of the reference, still more highly preferred embodiments have 90% to 110% of the activity of the reference.

Further preferred embodiments in this regard provide derivatives of the aforementioned prothrombin and prothrombin related polypeptides that have high specific thrombotic activity, particularly high specific activity as determined by activated partial thromboplastin time assay, especially using a standard human plasma preparation for comparison. Especially preferred in this regard are derivatives of prothrombin and prothrombin-related polypeptides comprising a region having an amino acid sequence with a degree an aforementioned degree of identity to the amino acid sequence of naturally occurring human prothrombin. Among the most especially preferred in this regard are those comprising a region having the amino acid sequence of human thrombin. Especially preferred in this regard are prothrombins having the amino acid sequence of naturally occurring human prothrombin.

In this regard especially preferred embodiments are those that have 50% or more of the thrombotic specific activity of a standard reference preparation of thrombotically active human plasma-derived prothrombin, particularly as measured by activated partial thromboplastin time assay. Particularly highly preferred embodiments in this regard have 65% or more of the specific activity of the aforementioned reference preparation, yet more highly preferred embodiments in this regard have 75% or more of the specific activity of the reference, preferably 85% or more, yet more preferably 90% or more, still yet more preferably 95% or more of the specific activity of the reference preparation. Also among highly particularly preferred embodiments in this regard are those with a higher specific activity than that of the reference preparation, particularly those with a substantially higher specific activity.

Other particularly preferred embodiments in this regard have 50% to 150% of the specific activity of the aforementioned reference preparation, particularly highly preferred embodiments in this regard have 60% to 125% of the specific activity of the reference preparation, yet more highly preferred embodiments have 75% to 110% of the specific activity of the reference preparation, still more highly preferred embodiments have 85% to 125% the specific activity of the reference, still more highly preferred embodiments have 90% to 110% of the specific activity of the reference.

Among preferred embodiments in this regard are derivatives that differ in post-translational modification from that found in human prothrombin prepared from natural sources. Especially preferred in this regard are differences that do not cause contraindications when administered to animal or human patients.

Also among particularly preferred embodiments in this regard are derivatives that have a lower or a higher content or different pattern of γ-carboxylation than that of normal human prothrombin isolated from sera, particularly normal sera and that typical of normal human prothrombin; but, that also are substantially indistinguishable from it, particularly as determined by USFDA regulatory practice, particularly those that have a lower content.

Also among particularly preferred embodiments in this regard are derivatives that have a lower or a higher fucose content than that of normal human prothrombin isolated from sera, particularly normal sera, and that typical of normal human prothrombin; but, that also are substantially indistinguishable from it, particularly as determined by USFDA regulatory practice, particularly in this regard those that have a higher fucose content.

Additionally among particularly preferred embodiments in this regard are derivatives that have a lower or a higher N-acetylgalactosamine content than that of normal human prothrombin isolated from sera, particularly that of normal sera and that typical of normal human prothrombin; but, that also are substantially indistinguishable from it, particularly as determined by USFDA regulatory practice, particularly those that have a higher content of N-acetylgalactosamine.

Further among particularly preferred embodiments in this regard are derivatives that have two or more of: (1) a lower or higher fucose content; (2) a lower or higher N-acetylgalactosamine content or (3) a lower or a higher content or different pattern of γ-carboxylation than that of human prothrombin isolated from sera, particularly that of normal sera and that typical of normal human prothrombin; but, that also are substantially indistinguishable from it, particularly as determined by USFDA regulatory practice, particularly those that have a higher content.

As to all of the aforementioned derivatives relating to fucose, N-acetylgalactosamine, and γ-carboxylation, preferred activities, in particular, are those percentages and ranges set out herein above.

### DNAs ENCODING PROTHROMBIN AND RELATED POLYPEPTIDES

Genetic constructs that encode prothrombin and prothrombin-related polypeptides for use in making transgenic organisms in accordance with the invention can be obtained using standard molecular biology techniques, including but not limited to techniques for cloning, synthesizing and modifying DNAs, RNAs, PNAs and combinations thereof, among others. Genomic, minigenes and cDNAs are particularly preferred in this regard.

Genetic constructs, such as genomic, minigenes or cDNA constructs, encoding prothrombin and related polypeptides derived from a variety of organisms may be used in the invention in this regard. For instance, genetic constructs encoding prothrombin and related polypeptides that can be used in the invention include, among others, those derived from genes and cDNAs of mammals, particularly mouse, rat, pig, sheep, goat and cow. Also preferred are those derived from genes and cDNAs of primates, especially chimpanzees. Most highly preferred are those derived from genes and cDNAs of humans.

Particularly preferred genetic constructs for use in the present invention are those that engender expression of human prothrombin-related polypeptides, especially those that encode human prothrombin itself. Genomic, minigenes and cDNAs are preferred in some embodiments in this regard. Genomic DNAs that encode human prothrombin can be obtained, for instance, from libraries of human genomic DNA using probes based on the published DNA sequence of human prothrombin and standard library screening and cloning techniques. Human cDNAs encoding prothrombin, for another example, can be obtained from cDNA libraries made from liver using much the same screening techniques and much the same probes as for human genomic DNAs. Minigenes can be constructed from genomic and/or cDNAs.

Cloned genes for human prothrombin, and for prothrombin of other origins, suitable for use in the invention include those described in Degen (Seminars in Thrombosis and Hemostasis 18(2): 230-242 1992), which is incorporated herein by reference in its entirety particularly as to the foregoing in parts pertinent to cloned prothrombin genomic DNAs, minigenes DNAs and cDNAs. Genetic constructs that engender production of naturally occurring prothrombin and prothrombin-derived and/or related polypeptides are highly particularly preferred in some aspects and preferred embodiments of the invention. Genetic constructs that engender production of altered, mutated, and/or modified forms of prothrombin and prothrombin-derived and or related polypeptides are preferred in other aspects and preferred embodiments of the invention.

Modifications can be introduced into naturally occurring prothrombin genes and polypeptides encoded thereby by techniques well known to the art, such as the synthesis of modified genes by ligation of overlapping oligonucleotides, and by introducing mutations directly into cloned genes, as by oligonucleotide mediated mutagenesis, inter alia.

Particularly preferred modifications in this context include but are not limited to those that alter post-translational processing as discussed above, that alter size, that fuse portions of other proteins to those of prothrombin, that alter the active site of the prothrombin, that stabilize the prothrombin or prothrombin-related polypeptide, that control transport and/or secretion of the polypeptide, that alter, augment, multiply, decrease or eliminate physiological activities of prothrombin or thrombin.

For instance, among modifications preferred in this regard are those that alter parts of the prothrombin or prothrombin-related polypeptide that do not alter thrombin or thrombin-related polypeptides derived from it, such as those generated by activation. Other preferred embodiments in this regard relate to modification that affect activation of prothrombins or prothrombin-related polypeptides by the natural series of proteolytic cleavages that occur during physiological activation, such as alteration to the sites of cleavage by Factor Xa complex. In this regard see, for instance, pages 514-516 in TEXTBOOK OF HEMATOLOGY, 2nd Edition, Shirlyn B. McKenzie, William & Wilkins, Baltimore (1996)) which is herein incorporated herein by reference in parts pertinent to thrombin and prothrombin, especially as to activation by proteolytic cleavage.

Further preferred embodiments in this regard relate to modifications that affect, alter, add to, or eliminate one or more of post-translational modifications of polypeptides of the invention. Certain particularly preferred embodiments in this regard relate to modifications that alter physiological functions and provide improved performance, such as improved thrombotic activity, improved stability, improved properties for purification, and improved physiological persistence, among others.

Certain preferred embodiments in this regard relate to addition, deletion or alteration of sites to change the γ-carboxylation of polypeptides of the invention. Particularly preferred embodiments in this regard relate to alterations about or to glutamic acid residues 7, 8, 15, 17, 20, 21, 26, 27, 30 and 33 (referring to the sequence of human prothrombin). Particularly preferred embodiments in this regard also relate to alterations that change γ-carboxylation at these sites or other sites and thereby improve calcium-dependent membrane binding, and or the ability of the prothrombin or prothrombin-related polypeptide to localize at sites of injury, and or improve the contribution of glutamic acid residues to modulating interaction and complex formation of prothrombin and prothrombin-related polypeptides with other vitamin K-dependent coagulation factors.

Certain preferred embodiments in this regard relate to addition, deletion or alteration of sites to change glycosylation of polypeptides of the invention. Particularly preferred embodiments in this regard relate to alterations to N-linked glycosylation sites at: Asn-79, Asn-101 and Asn-378, and the Asn-Leu-Ser site at Asn-165 which matches the consensus Asn-X-Ser/Thr sequence of N-linked glycosylation; and other such sites in prothrombins from non-human polypeptides. Such sites are described, for instance, in Degen, Seminars in Thrombosis and Hemostasis 18(2): 230-242 (1992) which is incorporated herein by reference in its entirety, as to the foregoing particularly with regard to glycosylation sites and consensus glycosylation sequences in prothrombins.

Particularly preferred embodiments in this regard arc those that improve glycosylation-dependent activities of polypeptides of the invention, such as physiological activities, including but not limited to enzymatic activity, substrate preferences, binding to cofactors and other moieties, complex formation, thermal stability, resistance to proteases and physiological persistence, among other things. In this regard see, for instance, PROTHROMBIN AND OTHER VITAMIN K PROTEINS Vols I and II, Seegers and Walz, Eds., CRC Press, Boca Raton, Fla. (1986) which is incorporated herein by reference in its entirety, as to the foregoing particularly in parts pertinent to glycosylation of prothrombin, especially in this regard Vol. 1, Chapter 8, Kobata and Mizuochi, Current Status of Carbohydrate Constituents and Prospects, 81-94.

### CIS-ACTING SEQUENCES FOR TRANSGENIC EXPRESSION

A wide variety of genes have been expressed in a wide variety of transgenic organisms. Many blood proteins in particular have been expressed in animals. Moreover, transgenic expression of blood proteins has been targeted to specific compartments. The cis-acting controls used in the past to express blood proteins in transgenic organisms also are useful, in many cases, in expressing prothrombin in transgenic organisms in accordance with the present invention. Examples in this regard are described in Lubon et al., Transfusion Medicine Reviews X(2): 131-141 (1996) which is incorporated by reference herein in its entirety. Some preferred embodiments relating to expression-regulatory regions for transgenic expression of prothrombin and or prothrombin-related polypeptides are described in further detail below.

In situ Activation

In certain instances, endogenous genes encoding prothrombin or prothrombin-related polypeptides (including those introduced into a transgenic animal) can be activated in situ in the genome by the introduction of exogenous sequences that effectuate the expression of the gene sequences already present in the genome. Such gene sequences can be native or they can be introduced and become integral to the genome. Thus, cells that express a given protein can be engineered by in situ alteration of endogenous DNA sequences. Techniques in this regard are described in, for example, WO 93/09222, WO 91/12650, and U.S. Pat. No. 5,641,670 each of which incorporated herein by reference in its entirety in parts pertinent to in situ activation methods for use in the present invention. As described therein and elsewhere, specific polynucleotide sequences corresponding to regions of a target gene, such as prothrombin or prothrombin-related gene, or to regions proximal or distal thereto are used to target integration of an exogenous construct into a specific site in a genome by homologous recombination of the specific sequences in the construct with their counterparts in the target site. Specific expression-regulatory sequences can be integrated into genomes in this way to control expression of specific genes, such as genes for prothrombin and related polypeptides. The methods can used to turn targeted genes on or turn them off or to alter their regulation in a call. Accordingly, these methods can be used to engender a prothrombin-related protein can be produced in cells not normally producing it, or to increase expression in cells that normally produce it at low levers. These methods also can be used to introduce specific mutations into a gene. By these means specific mutations can be introduced into coding regions of endogenous genes, such as those that encode functional regions of the protein.

In some cases, cells that can be manipulated in this way can be used to make transgenic organisms, although such methods are not available currently for all organisms. In one embodiment in this regard, for example, a DNA encoding a human prothrombin-related polypeptide can be introduced into a transgenic animal and subsequently modified therein as described above. Alternatively, a cell can be thus modified in vitro to express a prothrombin-related protein. Subsequently the cell can be introduced into the mammary tissue of an animal so that the prothrombin-related protein is secreted into the milk of the animal. Alternatively, a human cell derived, for example, from human mammary tissue, and compatible with growth and expression in animal mammary tissue, can be modified by means of homologous recombination in situ to express the prothrombin gene, a gene not normally expressed in such human cell. Such a gene can be further modified in situ to express a more highly beneficial or desirable prothrombin-related sequence mutant according to the present invention.

In an illustrative embodiment in this regard, the host cell can be a fertilized oocyte or embryonic stem cell that can be used to produce a transgenic animal containing the prothrombin-related gene. Alternatively, the host cell can be a stem cell or other early tissue precursor that gives rise to a specific subset of cells that can be used to produce transgenic tissues in an animal.

Promoters and Related Sequences

The cis-acting regulatory regions useful in the invention include the promoter used to drive expression of a gene in a transgenic organism effective for the production in the organism of prothrombin and prothrombin-related polypeptides. Preferred in this regard are regulatory regions that engender the production of significant amounts of prothrombin and or related polypeptides that can be recovered from the organism, purified and, in preferred embodiments, activated to provide thrombotic activity. The term “engender” refers to the case in which the regulatory regions are operably linked to the sequences to be expressed (coding sequences) prior to introduction into a cell. The term also encompasses the case in which an exogenous regulatory sequence is introduced into a cell, which sequence then integrates into the genome by homologous or nonhomologous recombination in such a manner as to become operably linked to an endogenous expressible sequence, such as a prothrombin-related coding sequence, and cause expression of or contribute to causing the expression of (as by an enhancer sequence) a desired endogenous sequence (preexisting in the genome prior to introduction of the exogenous sequence)

By “significant” is meant that the prothrombin and or prothrombin-related polypeptides can be recovered from the transgenic organism in amounts useful for research or for commerce or both. Preferred concentration ranges of the prothrombin-related polypeptide in milk, especially useful for purification for various purposes, extends from approximately 0.1-10 g/L, particularly 0.1-5 g/L. A preferred subrange includes from approximately 1-5 g/L. An even more preferred range includes from approximately 0.5-2.5 g/L. It is understood, however, that the concentration range in milk useful for purification will depend upon, for example, the animal in which the protein is produced. Accordingly, these ranges are not intended to be limiting but to provide guidance to preferred parameters.

Particularly preferred are regulatory regions that provide for the production of significant amounts of prothrombin and or prothrombin-related polypeptides in specific compartments of an organism, i.e., amounts that can be recovered in useful quantities. Particularly preferred compartments in this regard are compartments that accumulate and or store proteins. Also among preferred specific compartments are particular tissues or organs, including but not limited to liver, kidney, spleen, lymph node, peritoneum and small intestines. Especially preferred compartments in this regard are bodily fluids, such as lymph, saliva, blood and milk. Particularly especially preferred are blood and milk, most particularly milk.

Particularly useful regulatory regions for expression in milk are promoters that are active in mammary tissue, especially those that are specifically active in cells of mammary tissue, i.e., are more active in mammary tissue than in other tissues under physiological conditions where milk is synthesized. Most preferred are promoters that are both specific to and efficient in cells of mammary tissue. By “efficient” is meant that the promoters are strong promoters in mammary tissue that engender the synthesis of large amounts of protein, particularly for secretion into milk, especially milk of pigs.

Promoters and methods for producing proteins in milk of transgenic mammals that can be used in accordance with preferred embodiments of the invention in this regard are described in, for instance, U.S. Pat. No. 4,873,316 of Meade et al. on Isolation of Exogenous Recombinant Proteins From The Milk of Transgenic Mammals; U.S. Pat. No. 5,880,327 of Lubon et al. on Transgenic Mammals Expressing Human Coagulation Factor VIII; and U.S. Pat. No. 5,831,141 of Lubon et al. on Expression of a Heterologous Polypeptide in Mammary Tissue of Transgenic Nonhuman Mammals Using a Long Whey Acidic Protein Promoter, each of which is incorporated herein by reference in its entirety regarding the foregoing particularly in parts pertinent to transgenic production of polypeptides in milk of transgenic non-human mammals.

Whey acidic protein (referred to as “WAP”) promoters are among the most highly preferred promoters in this regard. Regulatory elements of the murine WAP gene are entered in GenBank (U38816) and cloned WAP gene DNAs are available from the ATCC. A variety of transcription-promoting WAP fragments, vectors, cloning methods and the like are described on the NIH mammary expression website at:

http://mammary.nih.gov/tools/molecular/Wagner001/WAP_vectors.htm

and in, among others, Campbell et al., Nucleic Acids Res. 12: 8685 (1984); Burdon et al., J. Biol. Chem. 266: 6909-14 (1991); Lakso et al., Proc. Nat'l Acad. Sci., USA 89: 6232-26 (1992); McKnight et al., Molec. Endocrin. 9: 717-724 (1995); Orban et al., Proc. Nat'l Acad. Sci., USA 89: 6861-65 (1992); Lubon et al., U.S. Pat.No. 5,880,327, Transgenic Mammals Expressing Human Coagulation Factor VIII; and Lubon et al., U.S. Pat. No. 5,831,141, Expression of a Heterologous Polypeptide in Mammary Tissue of Transgenic Nonhuman Mammals Using a Long Whey Acidic Protein Promoter, all of which are herein incorporated by reference in their entirety, particularly regarding the foregoing in parts pertinent to WAP cis-acting transcription elements useful for the production of polypeptides in transgenic organisms, particularly in mammary gland cells and milk of transgenic non-human female mammals. Among the most preferred promoters are those that regulate a whey acidic protein (WAP) gene, particularly, the murine and the rat WAP promoter. Especially preferred in this regard is the mouse “long” WAP promoter. In preferred embodiments, the WAP promoter is used in pigs, mice, rats, and rabbits.

Promoters of casein, lactalbumin and lactoglobulin genes also are preferred in certain embodiments of the invention in this regard, including, but not limited to the α-,β-, and γ-casein promoters and the α-lactalbumin and β-lactoglobulin promoters (“BLG promoters”), and derivatives thereof. Preferred among these promoters are those from rodents, especially mice and rats, and from pigs and sheep, especially the rat β-casein promoter and the sheep β-lactoglobulin promoter. In other preferred embodiments, the BLG promoter is used in pigs and sheep and the bovine casein promoter is used in mice and cows.

It is understood that the invention encompasses both constitutive and inducible promoters. Among preferred promoters in this regard are inducible promoters, particularly those that are inducible in mammary tissue, such as those induced by or during lactation. Among such promoters are the promoters for milk proteins such promoters of whey acidic protein genes. Such lactation-inducible promoters can be induced in organisms by inducers of lactation such as, for example, in many organisms, prolactin. A wide variety of constitutive and inducible promoters are know that can be used in this regard, including as well as those above, those that can be induced by hormones, ligands and metals. A variety of such promoters, their inducible elements, and their induction are described in for example, Sambrook et al. Molecular Cloning: A Laboratory Manual, 2^{nd} Ed., Cold Spring Harbor Laboratory Press, Cold Spring Harbor, N.Y. (1989), which is herein incorporated by reference in its entirety in part pertinent to promoters and induction.

Among the sequences that regulate transcription that are useful in the invention, in addition to the promoter sequences discussed above, are enhancers, splice signals, transcription termination signals and polyadenylation sites, among others. Particularly useful regulatory sequences include those that increase the efficiency of expression of prothrombin and or prothrombin-related polypeptides in transgenic organisms. Also particularly preferred in this regard are those that increase the specificity of expression of prothrombin and or prothrombin-related polypeptides in targeted compartments of a transgenic organism. Among highly particularly preferred regulatory regions in this regard are those that increase the efficiency, the specificity or both the efficiency and the specificity of expression of prothrombin or prothrombin-related polypeptides in mammary glands and in the milk of transgenic non-human mammals.

Especially useful in this regard are the other transcription regulatory sequences of genes expressed at high levels in mammary cells, such as those mentioned above, including but not limited to WAP genes, α-, β- and γ-casein genes, α-lactalbumin genes and β-lactoglobulin genes. Preferred sources for regulatory sequences in this regard include rodents (such as mice and rats), pigs and sheep.

Exemplary of additional preferred regulatory sequences are those associated with the mouse and rat WAP genes, rat β-casein genes and sheep β-lactoglobulin genes, respectively. The regulatory sequences most preferred for use in the present invention in this regard are those associated with whey acidic protein genes. Particularly preferred in this context are regulatory sequences of the murine whey acidic protein gene.

3′ Untranslated Sequences

Also among regulatory sequences preferred in certain embodiments of the invention are sequences comprised in the 3′ untranslated portion of genes that increase expression of transgenicly-encoded products particularly in mammary gland cells of transgenic non-human mammals, especially those that increase the amount of the product secreted into milk. Among highly preferred particular sequences in this regard are those that apparently stabilize mRNA transcribed from transgenes. Among preferred embodiments in this regard are sequences that comprise a polyadenylation signal. Among preferred regions of this type are those derived from the genes for proteins that are expressed at high levels in mammary gland cells and or encode proteins that are found at high concentrations in milk. Especially preferred in this regard are sequences of the 3′ untranslated region of whey acidic protein genes, particularly the mouse and rat whey acidic protein genes. Highly especially preferred is this regard are sequences of the long mouse and rat whey acidic protein promoter constructs.

Trafficking and Translational Signals

Also important to the invention are signal peptide sequences that direct secretion of proteins into the milk of transgenic animals. In this regard, both endogenous and heterologous signal sequences are useful in the invention. Generally, the signal peptides of proteins normally secreted into milk are useful in the invention. The signal sequences of proteins that occur in high concentration in milk are particularly preferred, such as the signal peptides of the whey acidic proteins, caseins, lactalbumins and lactoglobulins, including, but not limited to the signal peptides of the α-, β- and γ-caseins and α-lactalbumin and β-lactoglobulin. The signal sequences of whey acidic proteins are particularly preferred in this regard, especially the signal sequences of murine whey acidic proteins and the signal sequences of rat whey acid proteins. Also among preferred signal sequences in this regard are the signal peptides of secreted coagulation factors. Particularly preferred in this regard are the signal peptides of prothrombins and t-PAs, especially the secretion signal sequence of human prothrombin.

Among the sequences that regulate translation and transport, in addition to the signal sequences discussed above, are ribosome binding sites and sequences that augment the stability of mRNA. Especially useful are the translation regulatory sequences of genes expressed at high levels in mammary cells. For instance, the regulatory sequences of the α-, β- and γ-casein genes and the α-lactalbumin and β-lactoglobulin genes are preferred, especially those from rodents (mice and rats), pigs and sheep. Also particularly preferred are the regulatory sequences of rat β-casein and the sheep β-lactoglobulin genes.

Also, especially useful in the present invention are sequences that advantageously modulate post-translational modifications of transgenic prothrombin, and prothrombin-related polypeptides produced in accordance with the invention herein described.

### PURIFICATION OF CONSTRUCTS

Constructs for producing prothrombin and related polypeptides in accordance with the invention, such as double-stranded DNA and DNA:RNA hybrid constructs, can be prepared by any of a wide variety of well known molecular biology methods. DNAs in double-stranded form may be manipulated by conventional methods to provide constructs having the stnictures and properties set out above and elsewhere herein for expression of prothrombin and related polypeptides in transgenic organisms.

For DNA:RNA hybrids, well known vectors that contain bacteriophage promoters, such as the T3 and T7 promoter can be used to produce RNA for DNA:RNA hybrids and well known vectors that produce single-stranded DNA may be used to produce single-stranded DNA for DNA:RNA hybrids.

Constructs can be amplified by conventional techniques for cloning and propagation in a host organism such as a bacterial host, a yeast host, an insect cell host, or a mammalian cell host. Constructs also can be amplified by in vitro methods such as PCR. Constructs can be derived from natural, cloned or synthesized DNA or RNA in whole or in part. Polynucleotide constructs may contain modified bases as well as the bases that occur naturally in DNA and RNA.

Often constructs for making transgenic organisms in accordance with the invention are manipulated or propagated joined to or in the presence of other polynucleotides. These extraneous polynucleotides can be removed prior to using a construct to produce a transgenic organism. For instance, a construct that was propagated and amplified in a cloning vector typically can be separated from the vector by restriction enzyme cleavage and then purified.

Constructs for introduction into cells to make transgenic organisms in accordance with the invention can be purified by well known techniques. For instance, among other well know techniques that can be used, constructs can be purified by agarose gel electrophoresis and electroelution, by HPLC, by ultracentrifugation through a sucrose gradient, by ultracentrifugation through an NaCl gradient or, in certain particularly preferred embodiments in this regard, by combination of two or more of electroelution, HPLC, sucrose gradient centrifugation and NaCl gradient centrifugation.

### ORGANISMS

A wide variety of hosts can be used for transgenic production of prothrombin and prothrombin-related polypeptides in accordance with the present invention. Particularly preferred are those that provide prothrombin and/or prothrombin-related polypeptides with the post-translational modifications required for physiological activity. Especially preferred in this regard are those that provide high specific activity prothrombin and those that provide high yields of prothrombin. Most especially preferred in this regard are those that provide high yields of high specific activity prothrombin and/or prothrombin related polypeptides. Organisms that do not suffer adverse effects of transgenesis and or transgene expression are similarly preferred, as are those that do not suffer adverse effects of production, accumulation or harvesting of transgenicly expressed prothrombin and/or related polypeptides.

All lactating animals, that is, all mammals, particularly are preferred in this regard. Preferred mammals in this regard include domesticated mammals, particularly livestock animals. Particularly preferred mammals include mice, rats, hamsters, rabbits, pigs, sheep, goats, cows and horses. More particularly, mice, pigs, sheep and cows are preferred. Among the most preferred mammals at present are mice, pigs and sheep. Of these, pigs are especially particularly preferred.

### HARVESTING AND PURIFICATION

A wide variety of well known techniques may be employed to isolate and purify prothrombin and prothrombin-related polypeptides from transgenic organisms in accordance with the invention. Among preferred general purification methods in this regard are those described in Seegers et al., Preparation of Prothrombin Products, Biochemistry 6: 85-95 (1944), Seegers et al., Preparation and Properties of Thrombin, 194-201 (1968), Grant et al., Archive Biochem. Biophys 176: 650-662 (1975), as well as methods described in PROTHROMBIN AND OTHER VITAMIN K PROTEINS Vols I and II, Seegers and Walz, Eds., CRC Press, Boca Raton, Fla. (1986), which are incorporated herein by reference in their entirety, as to the foregoing particularly in parts pertinent to purification of prothrombin and thrombin, especially in this regard as to Seegers and Walz Vol. II, Chapter 10, Murano, Commercial Preparations of Vitamin K-Dependent Factors and Their Use in Therapy, 131-142.

Obtaining milk from a transgenic animal within the present invention can be accomplished by a variety of well know methods, such as those described in, among others, Burney et al., J. Lab. Clin. Med. 64: 485 et seq. (1964) and Velander et al., Proc. Nat'l Acad. Sci. USA 89: 12003 et seq. (1992) each of which is herein incorporated by reference in its entirety particularly regarding the foregoing in parts pertinent to obtaining milk from transgenic animals.

The prothrombin or prothrombin-related polypeptide contained in milk can be purified by known means without unduly affecting activity. Generally, it is preferred that prothrombin or prothrombin-related polypeptides in milk produced pursuant to the present invention should be isolated as soon as possible after the milk is obtained from the transgenic mammal, thereby to mitigate any deleterious effect(s) of milk components on the structure, properties or activities of the prothrombin or prothrombin-related polypeptide. Preferred methods include those that use one or more of cryoprecipitation, ion-induced precipitation, anion exchange, and/or immunochromatography to purify the prothrombin or prothrombin-related polypeptide form milk or whey. For the most part the methods are employed conventionally. Representative methods in this regard are described in, among others, Bringe et al., J. Diary Res. 56: 543 et seq. (1989) which is incorporated herein by reference in parts pertinent to methods that can be used in whole or part to purify prothrombin or prothrombin-related polypeptides from transgenic milk.

There are proteases in milk that may degrade proteins, such as transgenicly expressed prothrombin and prothrombin-related proteins. The main proteases in milk thus far identified are alkaline proteases with tryptic and or chymotryptic activities, a serine protease, a chymotrypsin-like enzyme, an aminopeptidase and an acid protease. Methods may be employed for isolation and purification of transgenic prothrombin or prothrombin-related polypeptides that prevent proteolytic degradation by endogenous milk proteases, such as those noted above. Among preferred methods in this regard are rapid processing of whole milk, the use of low temperatures that inhibit protease activity and or decrease degradation of transgene products in milk and the use of protease inhibitors. Specific inhibitors that may be useful in this regard are well known to those of skill, and are widely available from commercial reagent suppliers such as Sigma Chemical Company.

### YIELD

Prothrombin and prothrombin-related polypeptides expressed in transgenic organisms in preferred embodiments of the invention have a high percentage of active protein, as measured by conventional assays of prothrombin and or thrombin activity. Particularly, in preferred embodiments of the invention in this regard, not only do the prothrombin and prothrombin-related polypeptides expressed in and or obtained from the organisms contain a high percentage of protein having prothrombin activity, a high percentage of the polypeptides can be activated to a form that has the thrombotic and or other activities of thrombin. In preferred embodiments in this regard the activities and their determination are as described above.

In certain preferred embodiments in this regard, prothrombin and prothrombin-related polypeptides expressed in the mammary tissue and secreted into the milk of a transgenic mammal in certain preferred embodiments of the invention preferably have a high percentage of active protein, as measured by conventional assays of prothrombin and thrombin activity. Particularly, in preferred embodiments of the invention in this regard, not only do the prothrombin and prothrombin-related polypeptides secreted into the milk contain a high percentage of protein having prothrombin activity, a high percentage of protein can be activated to a form that has the thrombotic and or other activities of thrombin. In preferred embodiments in this regard the activities and their determination are as described elsewhere herein.

Yields of polypeptides of the invention in this regard in preferred embodiments are sufficiently high for recovery of useful amounts. In particularly preferred embodiments the yields are substantially better than those previously achieved by other methods, either as to concentration, total amount of polypeptide obtained, activity, specific activity or homogeneity, including homogeneity of activity, specific activity, physiological activity, general or specific post-translational modification, including but not limited to γ-carboxylation and glycosylation, or a combination of one or more of any of the foregoing, proteolytic processing and or activation, among others.

For milk of transgenic non-human mammals preferred embodiments of the invention in this regard have yields in the range of 0.05 to 5.0 g/L, especially 0.1 to 3 g/L, as well as activities, specific activities and the like of the preferred embodiments described in detail above.

### ACTIVATION

In some preferred embodiments of the invention transgenic prothrombin and or prothrombin-related polypeptides are produced in transgenic organisms in a thrombotically inactive form and then are activated to a thrombotically active form. Particularly preferred embodiments in this regard relate to transgenic thrombotically inactive prothrombins produced in transgenic organisms and their activation to thrombotically active thrombins, especially in this regard human prothrombin, produced in transgenic organisms and their activation to human thrombins.

Activation in this regard preferably is carried out after isolation of the prothrombin or prothrombin-related polypeptide from the organism. It may be carried out at any stage of purification thereafter, including at any time from immediate isolation to the point of end-use requiring thrombotic activity. Preferred embodiments in this regard relate to specific production and purification methods, specific storage and product needs, and specific applications and thus do not relate generally to any one particular time or method of activation.

Typically, as for native prothrombins and thrombins, activation involves cleaving the thrombotically inactive prothrombin or prothrombin-related polypeptide in one or more places to generate the active thrombin or thrombin-related polypeptide. The cleavage sites may be the naturally occurring sites for activation or they be different sites, such as sites introduced for this purpose or sites utilized by cleavage agents that do not ordinarily act on prothrombins in physiological settings. If cleavage at more than one site is involved the agents for cleavage at each site may be the same or may be different, and the different sites may be cleaved in a single reaction or in two or more successive reactions that occur and or can be controlled and or can be carried out independently of one another.

Cleavage may be carried out using one or more enzymes or enzyme complexes, such as the enzymes that naturally cleave prothrombin during physiological activation, or by chemical methods, among others. Among preferred enzymatic activation methods are those that use thrombins.

Also among preferred enzymatic activation methods are those that use Factor Xa. Useful methods in this regard are described in, among others, Rosing et al., J. Biol. Chem. 261(9): 4224-4228 (1986); Krishnaswamy et al., J. Biol. Chem. 261(19): 8977-8984 (1986); Boscovic et al., J. Biol. Chem. 265(18): 10497-1010505 (1990); Tans et al., J. Biol. Chem. 266(32): 21864-2873 (1991); Tijburg et al., J. Biol. Chem. 266(6): 4017-4022 (1991; and Walker et al., J. Biol. Chem. 269(44): 27441-227450 (1994) each of which is incorporated herein by reference in its entirety in parts pertinent to activation of prothrombins and prothrombin-related polypeptides by Factor Xa cleavage.

Additionally among preferred enzymatic activation methods are those that use venom proteases. Methods useful in this regard are described in, among others, Franza et al., J. Biol. Chem. 250(7): 7057-7068 (1965) and Rhee et al., Biochemistry 21: 3437-3443 (1982) each of which is incorporated herein by reference in its entirety in parts pertinent to activation of prothrombins and prothrombin-related polypeptides by cleavage with venoms. Further among preferred enzymatic activation methods are those that utilize endogenous enzymes, such as activating enzymes that occur in milk.

Chemical methods also can be used for activation and in some aspects and embodiments of the present invention are preferred for large scale preparations. However, enzymatic methods also may be used for large scale processes and chemical methods can be used for small preparations as well. Among highly particularly preferred chemical activation methods is activation using sodium citrate. Methods for sodium citrate activation useful in this regard are described in, among others, Seegers et al., Blood 5: 421-433 (1950), Heldebrandt et al., J. Biol. Chem 248(10): 3642-3652 (1973) and PROTHROMBIN AND OTHER VITAMIN K PROTEINS Vols I and II, Seegers and Walz, Eds., CRC Press, Boca Raton, Fla. (1986) especially in this regard Vol. I, Chapter 9, Seegers, Prothrombin and Factor X Activation in 25% Sodium Citrate Solution and Related Phenomena, 95-101, each of which is incorporated herein by reference in its entirety in parts pertinent to activation of prothrombins and prothrombin-related polypeptides by sodium citrate. Another particularly preferred chemical activation method useful in this regard is activation using protamine sulfate. Protamine activation methods useful in this regard are described in Miller, Ann. N.Y. Acad. Sci. 320 336-342 (1981) which is incorporated herein by reference in its entirety in parts pertinent to activation of prothrombins and prothrombin-related polypeptides by protamines. Yet another preferred chemical activation method useful in this regard is activation using polylysine. Polylysine activation methods useful in this regard are described in Miller, J. Biol. Chem 236: 63-64 (1960) which is incorporated herein by reference in its entirety in parts pertinent to activation of prothrombins and prothrombin-related polypeptides by polylysine.

### ASSAY OF PROTHROMBIN AND THROMBIN AMOUNTS AND ACTIVITIES

A variety of well known and widely employed thrombin and prothrombin activity assays can be used to determine activities of transgenic compositions and polypeptides of the invention. Among these are clotting assays, antigen assays, membrane-dependent activity assays, calcium-dependent assays, membrane and calcium-dependent assays and chromogenic assays, to name a few. Among preferred assays in the present invention are the APTT assay, ELISA assay and chromogenic assay of amidolytic activity. Preferably, a reference standard preparation is used to accurately quantify amounts and activities. A variety of standard preparations of prothrombin and thrombin are available that can be used for this purpose, including standards used by clinical laboratories, such as those from commercial suppliers of clinical laboratory reagents of this type. One preferred source of reference standard preparations of this type is the NIH, non-commercial sources and commercial vendors of NIH references preparations.

Prothrombin activity in a sample often is determined by activating prothrombin in a sample and then determining thrombin enzymatic activity, generally by lysis of chromogenic substrate. For instance, as described in U.S. Pat. No. 5,811,279, which is incorporated by reference herein in its entirety in parts pertinent to assay of prothrombin and thrombin activity, activation of thrombin activity in prothrombin-containing samples can be determined by incubating samples for a fixed time period with Factor X (120.5 mμg/ml Factor X in 1 mM EDTA and PEG 4,000, pH 7.4 at 25° C., then incubating the sample for a period of time with a chromogenic substrate for thrombin activity, such as S-2238, and then determining the amount of color generated in each reaction. Dilutions of each sample generally are performed for quantitative results and the amount of thrombin activity in or activated in each sample generally is interpolated from a graph of color density versus amount of a reference standard thrombin preparation, preferably calibrated to the US Standard Thrombin so that the amount of thrombin activity can be expressed in NIH units (one NIH plasma unit of prothrombin activity is the prothrombin activity in 1 plasma unit of normal plasma.

Clotting assays also may be used to determine prothrombin activity. Clotting assays typically measure the clotting activity of thrombin in a sample after treatment to activate prothrombin. As described in U.S. Pat. No. 5,445,958, for instance, which is incorporated herein by reference in its entirety in parts relating to prothrombin assay and thrombin assay. Unitage of the clotting activity determined by such assays typically is defined in terms of Working Standards such as Working Standard 87/532 which was calibrated against the 1st International Standard for Factors II concentrates and provides for comparison of different assays to one another.

The amount of prothrombin in samples can be measured by a variety of methods, many of which employ prothrombin and or thrombin specific antibodies or antibody-derived antigen-determining reagents. Determining the amount of prothrombin or thrombin in a sample can be carried out in solution, by ELISA, using gel electrophoresis, Western blotting, HPLC and or other separation techniques, which are well known and widely used in research and clinical laboratories, Determination of the amount of prothrombin and or thrombin in a sample can be combined in variety of ways with measures of activity in determining specific activities.

Many methods for determining prothrombin and thrombin amount and activities in samples rely on standards for controls and standard curves for interpolations. Human thrombin and prothrombin samples are available from a variety of suppliers. A few examples are described below. Human Prothrombin (Factor II) prepared from fresh frozen human plasma is available in 20 mM Tris-HCl/0.1 M NaCl/1 mM Benzamidine/pH 7.4 as a homogeneous preparation (as judged by 10% SDS-PAGE gels) that shows no reduction upon incubation with 2-mercaptoethanol, having an Extinction Coefficient (1%) of 13.6, a specific activity of 1 unit/90 μg, and a molecular weight of 72,000 daltons. Human Thrombin (Factor IIa) prepared from homogeneous human prothrombin by activation with Factor Xa, Factor Va, and phospholipid is available as a homogeneous preparation (as judged by 10% SDS-PAGE gel electrophoresis) with a minimum activity of 2,700 NIH units/mg compared to NIH standard thrombin. The preparation is supplied in 50 mM Sodium Citrate/0.2 M NaCl/0.1% PEG-8000/pH 6.5, with an Extinction Coefficient (1%) of 18.3, and a molecular weight of 37,000 daltons. A variety of other reagents and standards useful for determining thrombin and prothrombin amounts and activities are well known and widely available as well.

### USES

Prothrombins and prothrombin-related polypeptides and the thrombins and thrombin- related polypeptides of the invention have many uses, including both clinical and non-clinical applications; that is, medically related uses, including medical-related uses for both non-human and human subjects, and uses that are not medically related.

Certain highly preferred embodiments of the invention relate is this regard to uses of the thrombotic activity of the thrombins and thrombin-related polypeptides of the invention, derived from the prothrombins and prothrombin-related polypeptides of the invention. Particularly preferred embodiments in this regard relate to the prothrombins, prothrombin-related polypeptides, thrombins and thrombin-related polypeptides that provide thrombotic activity useful in veterinary and human clinical circumstances. Among clinically important preferred applications in this regard are uses of the same to promote hemostasis, to improve anastomoses, to control hemorrhage, to achieve good hemostasis on bone defects, to seal vascular prostheses, to seal lesions and stumps, to treat pleurodesis, to close fistulas, to seal membranes, in procedures to extract stones and to prevent or reduce perioperative bleeding, to mention just a few. Additional uses in this regard are set out, for instance, in PROTHROMBIN AND OTHER VITAMIN K PROTEINS Vols I and II, Seegers and Walz, Eds., CRC Press, Boca Raton, Fla. (1986) which is incorporated herein by reference in its entirety, as to the foregoing particularly in parts pertinent to uses of prothrombin and thrombin, especially in this regard Vol. II, Chapter 7, Deutsch, The Clinical Use of Thrombin, 92-103.

Particularly preferred embodiments in this regard especially relate to uses to promote hemostasis per se. Among a wide variety of such uses preferred embodiments relate to using thrombotically active polypeptides of the invention to, among other things, promote hemostasis in animals and humans, particularly at, to name a few, lacerations and other wounds, sites of organ rupture, sites of bleeding during surgery, burn sites, sites of traumatic injury, surgical sites such as partial resections, including partial brain resections, bleeding biopsies, sites of tumor extirpation, including tumors from parenchymatous organs such as liver, spleen, pancreas, kidney, brain and prostate gland among others, sites of donations of skin grafts, sites of skin grafts, extraction of teeth sites, nose bleeding, sinus bleeding, bleeding in or near bones, gastrointestinal bleeding, and conjunctival wounds, to name but a few.

In this regard a preferred use relates to uses to promote anastomoses, particularly and to, among other things, tighten classically sutured anastomoses, to reduce the number of sutures in, for example, anastomoses of intestines, small vessels, maxillo-facial vessels and extracranial anastomoses, to prevent kinking of arterial grafts, and to promote the combination of nerve endings, to name but a few.

Certain very highly particularly preferred embodiments in this regard relate to the use of prothrombins, prothrombin-related polypeptides, thrombins and thrombin-related polypeptides of the invention to promote hemostasis in surgery and of wounds associated with trauma, particularly wounds in civilian and military personnel that result from warfare.

With regard to these and other uses not mentioned preferred prothrombins, prothrombin-related polypeptides, thrombins and prothrombin-related polypeptides are as described above concerning, inter cilia, structure, activation, activity, modification and the like.

Among particularly highly preferred embodiments in this regard are uses of prothrombins, prothrombin-related polypeptides, thrombins and thrombin-related polypeptides of the invention especially in regard to control of bleeding by fibrin glues, particularly but not limited to uses as or in fibrin glue compositions and or formulation and or in devices for using and administering fibrin glues such as for instance creams, lotions, pastes, salves, liquids, bandages, gauzes, swabs, applicator packs and the like.

The present invention is further described by reference to the following examples which are provided by way of illustration only and do not themselves depict in their particulars or in any general fashion limitations of the present invention.

### EXAMPLE 1

### Construction of DNAs Useful for Transgenic Expression of Prothrombins in Milk

As illustrated below, DNAs, vectors and expression constructs for use in accordance with the invention can be made using standard recombinant DNA techniques, such as those set forth in MOLECULAR CLONING, A LABORATORY MANUAL, Vol. 1-3, Sambrook et al., Cold Spring Harbor Press (1989), which is incorporated herein by reference in its entirety.

Using such methods, the cis-acting expression signals of the mouse “long WAP” promoter construct are operatively fused to DNAs encoding human prothrombin for introduction into and expression in transgenic mice and pigs. WAP genes and promoters therefrom are obtained and are as described in the foregoing references on WAP genes and promoter sequences, particularly U.S. Pat. No. 5,880,327 of Lubon et al. for Transgenic Mammals Expressing Human Coagulation Factor VIII, and U.S. Pat. No. 5,831,141 of Lubon et al. for Expression of a Heterologous Polypeptide in Mammary Tissue of Transgenic Nonhuman Mammals Using a Long Whey Acidic Protein Promoter, each of which are herein incorporated by reference in their entirety, as to the foregoing particularly in parts pertinent to obtaining the murine WAP gene, especially long WAP promoter-containing fragments for expressing a prothrombin or a prothrombin-related polypeptide in milk of a transgenic mammal.

In particular, the vector MCS of pUC19 is replaced by a NotI site. A mouse genomic fragment containing the WAP promoter and extending upstream about 4.6 kb from a point near but upstream of the WAP translation start site is obtained. A second mouse genomic fragment containing about 1.3 kb of the WAP gene immediately downstream of the translation stop site also is obtained. The two fragments are joined to form a unique KpnI at the fusion site. The resulting fragment is cloned into the NotI site in the MCS-replaced pUC19 vector. Prothrombin-encoding DNAs are inserted into the KpnI site.

A full-length human prothrombin sequence in the GenBank database (Accession Number J00307) is used to design probes that can be used to obtain DNAs encoding human prothrombin by conventional means by screening a human liver cDNA library or a human genomic DNA library and purifying therefrom a full-length prothrombin cDNA using methods much the same as those described by MacGillivray et al., Ann. N.Y. Acad. Sci. 485: 73-79 (1986); Jorgensen et al., Circulation 24(Supp2): 1637 (Abstract) (1986); Degen et al., DNA Cell Biol. 9: 487-498 (1990); Degen et al., Biochemistry 22: 2087-2097 (1983); and U.S. Pat. No. 4,476,777 of Holly et al. on Methods for Producing Thrombin, each of which is incorporated herein by reference in its entirety, particularly as to the foregoing in parts pertinent to obtaining DNAs encoding prothrombin and related polypeptides, especially genomic and cDNAs encoding the full length of human prothrombin.

Human prothrombin-specific probe sequences are designed using the GenBank full length human prothrombin sequence and standard software. The probe sequences are used to search the dbEST database to identify the most full length human prothrombin-encoding cDNA clone in the IMAGE consortium library. The longest clone identified is IMAGE clone ID 74521 based on (1) dbEST Id:113104; EST name: yb49f01.s1; GenBank Ace: T59037; GenBank gi 660874; GDB Id:496186 and (2) dbEST Id:113178; EST name: yb49f01.r1; GenBank Acc: T59111; GenBank gi 660948, GDB Id:496186.

In addition, oligonucleotides having the human prothrombin-specific probe sequences are synthesized and purified by conventional means.

The aforementioned IMAGE clone is picked from the IMAGE library and streaked to obtain single colonies. Individual colonies are picked and verified as prothrombin DNA-containing clones by hybridization with labeled human prothrombin-specific probe oligonucleotides.

The cDNA is isolated from a positive and sequenced. The sequence is that of the GenBank human prothrombin sequence in, except for about 400 by missing from the 5′ end. The missing portion of the cDNA is recovered by specific synthesis of cDNA using a prothrombin cDNA-specific primer designed from the 5′ most prothrombin sequence of the incomplete IMAGE clone. The missing region is cloned directly into a standard plasmid vector for later mating to the short IMAGE clone. The missing region also is recovered by PCR using a second primer specific for the cDNA 5′ end at the translation start site. Finally, a full length human prothrombin cDNA is recovered directly from the cDNA library using a second primer specific to the 3′ end of the cDNA. The missing fragment, recovered by either method is joined with the cDNA from the IMAGE clone to form a full length prothrombin-encoding cDNA. For PCR of the full length clone directly, both primers have a KpnI site upstream of the priming sequence and, after KpnI cleavage, the full length cDNA recovered by PCR is inserted into the KpnI-digested long WAP construct.

(1) WAP-huPTc-01

A DNA construct called WAPhuPTc01 is made by inserting into the human prothrombin cDNA into the unique KpnI site of the murine long WAP DNA, 24 base pairs 3′ to the transcriptional start site. The WAP-prothrombin product is then ligated into a Bluescribe vector (Stratagene) to facilitate further manipulation.

(2) WAP-huPT-c02

Another DNA construct called WAP-huPT-c02 is made using similar methods. It is much the same as WAP-huPT-01, comprising the same murine WAP and human prothrombin DNAs, but lacking sequences artefactually present in WAP-huPT-01 as a result of cloning procedures.

(3) WAP-huPT-g01

Similar methods are used to obtain a full length genomic clone for human prothrombin. However, whereas the cDNA sequence does not contain a KpnI site, the genomic DNA sequence contains a KpnI. As a result the genomic expression is constructed by first removing the internal KpnI site or by using a partially digestion from which the full length prothrombin-genomic gene is recovered and inserted into the KpnI site of he long WAP promoter construct. In addition, the coding region of the genomic sequence is rather long, about 20,113 bp, and techniques appropriate to the manipulation of long DNAs must be employed. Accordingly, prothrombin-encoding human genomic DNA is identified in a Cal Tech human BAC library, and the prothrombin-encoding region is verified by PCR and direct sequencing. A single fragment containing the prothrombin-encoding region is isolated, modified as noted above to remove the internal KpnI site and to contain KpnI ends, cloned for stability in pBeloBAC11 and, either directly or after recovery form pBeloBAC11, cloned into the KpnI site of the long WAP promoter construct. In addition, the genomic DNA is recovered intact, joined to adaptors at both ends. partially digested with Kpnl and the KpnI fragment containing the intact gene is cloned into the WAP promoter construct at the KpnI site. A two fragment strategy also is used in which the two KpnI genomic fragments are generated by cleavage at the internal KpnI site, manipulated separately and then operatively recombined in correct orientation with one another in the KpnI site of the long WAP promoter construct.

### EXAMPLE 2

### Preparation of DNAs for Microinjection

The WAP-human prothrombin cDNA fragment for microinjection is prepared from WAP-huPT-02 as follows. The DNA for injection is severed intact from other parts of WAP-huPT-02 by restriction enzyme cleavage. The solution containing the WAP-huPT

DNA is brought to 10 mM magnesium, 20 mM EDTA and 0.1% SDS and extracted with phenol/chloroform. The DNA then is precipitated from the aqueous layer with 2.5 volumes of ethanol in the presence of 0.3 M sodium acetate at −20° C. overnight. After centrifugation, the pellet is washed with 70% ethanol, dried, and resuspended in sterile distilled water. The WAP-huPT DNA then is further purified by sucrose gradient centrifugation using standard procedures. DNA concentrations are determined by agarose gel electrophoresis by staining with ethidium bromide and comparing the fluorescent intensity of an aliquot of the DNA with the intensity of standards. Samples are adjusted to 10 μg/ml and stored at −20° C. prior to microinjection.

### EXAMPLE 3

### Transgenic Animal Production

(1) Mice

Transgenic mice that express human prothrombin are produced by pronuclear microinjection using standard techniques as described below.

Glass needles for micro-injection are prepared using a micropipet puller and microforge. Injections are performed using a Nikon microscope having Hoffman Modulation Contrast optics, with micromanipulators and a pico-injector driven by N₂ (Narashigi).

Fertilized mouse embryos are surgically removed from the oviducts of super-ovulated female CD-1 mice and placed into M2 medium. Cumulus cells are removed from the embryos by treatment with 300 μg/ml hyaluronidase. The embryos are rinsed after treatment in fresh M2 medium, transferred into M16 medium and stored at 37° C. prior to injection.

Female mice are made pseudo-pregnant by mating with vasectomized males. DNA is injected into the male pronucleus of embryos prepared as described above. The injected embryos are implanted into avertin-anesthetized pseudo-pregnant recipient females. Embryos are allowed to come to term and newborn mice are delivered. The newborn mice are analyzed for the presence and integration of the injected DNA.

(2) Pigs

DNAs and injection equipment and supplies are prepared much the same as described for mice. Embryos are recovered from oviducts obtained from healthy female pigs. They are placed into a 1.5 ml microfuge tube containing approximately 0.5 ml embryo transfer media (phosphate buffered saline+10% fetal calf serum, Gibco BRL) and centrifuged for 12 minutes at 16,000×g RCF (13,450 RPM) in a microcentrifuge (Allied Instruments, model 235C). The embryos are removed from the microfuge tube with a drawn and polished Pasteur pipette and placed into a 35 mm petri dish for examination. If the cytoplasm is still opaque with lipid such that pronuclei are not visible, the embryos are centrifuged again for 15 minutes. Embryos to be microinjected are placed into a microdrop of media (approximately 100 μl) in the center of the lid of a 100 mm petri dish. Silicone oil is used to cover the microdrop and fill the lid to prevent media from evaporating. The petri dish lid containing the embryos is set onto an inverted microscope (Carl Zeiss) equipped with both a heated stage and Hoffman Modulation Contrast optics (200× final magnification). A finely drawn (Kopf Vertical Pipette Puller, model 720) and polished (Narishige microforge, model MF-35) micropipette is used to stabilize the embryos while about 1-2 picoliters of purified DNA solution containing approximately 200-500 copies of DNA construct is delivered into the male pronucleus with another finely drawn micropipette. Embryos surviving the microinjection process as judged by morphological observation are loaded into a polypropylene tube (2 mm ID) for transfer into a recipient pseudo pregnant female pig.

### EXAMPLE 4

### Assessing Construct Integration

(1) Preparation of DNA from Transgenic from Mice and Pigs

A 5 mm piece of mouse tail is removed from young, potentially transgenic mice at weaning (3 weeks of age), minced, and treated with proteinase K and SDS at 37° C. overnight. The mixture then is incubated with DNase-free RNase at 37° C. for 1-2 hours. In some cases the mixture is extracted extensively with phenol/chloroform. DNA is precipitated from the mixture with sodium acetate and ethanol at −20° C. overnight, collected by centrifugation, washed in 70% ethanol and then is dried. The dried DNA pellet is used directly for PCR.

A similar procedure is used to prepare DNA from pigs.

(2) Oligonucleotide Probes for PCR Assay

Oligonucleotide pairs are used to prime polymerase chain reactions to detect WAP-huPT constructs in the transgenic animals. Oligonucleotide pairs that bridge the WAP-huPT DNA are used to detect the exogenously-derived prothrombin-encoding DNA in cells of the transgenic organisms.

A probe pair that targets a region in the WAP sequence 5′ of the KpnI site and a region in the endogenous mouse WAP sequence that lies 3′ of the KpnI site is used to provide a positive control in PCR assays of mice DNA.

(3) PCR Reaction Conditions and Product Analysis

PCR reactions are performed using 40 cycles in an automated temperature cycler (M.J. Research). An annealing temperature of 58° C., a denaturation temperature of 94° C., and an extension temperature of 72° C. 100 ng of oligo primers and 50 ng of (genomic) template DNA are used per PCR reaction. Products of the PCR reactions are analyzed by agarose gel electrophoresis. Fragments sizes are estimated by migration relative to molecular weight standards and compared with the sizes expected for the injected constructs.

(4) Results of PCR Analysis of Transgenic Animals

PCR analysis of potentially transgenic mice and pigs that developed from embryos microinjected with the expression constructs described above shows that injected constructs frequently are integrated into the embryonic genomes of both mice and pigs. PCR analysis of offspring shows Mendelian transmission of integrated transgenes in mice and pigs that are initially shown to have integrated the xenogenetic DNA constructs.

### EXAMPLE 5

### Preparation of Milk and Whey

(1) Mice

Lactating mice are milked an average of 3 times per week. The mice are first separated from their young for approximately 5 hours. Then they are anesthetized by injection of 0.4 ml avertin at 2.5% (I.M.). 0.2 ml oxytocin is administered at 2.5 IU/ml (I.P.) to stimulate the release of milk. A milking device consisting of a vacuum pump (2.5 psi) and syringe with an eppendorf tip is used to express milk from the animals and direct it into an eppendorf tube. The milk is kept on ice throughout the collection process.

To prepare whey the collected milk is diluted 1:1 (vol:vol) with TS buffer (0.03 M Tris pH 7.4; 0.06 NaCl) and centrifuged in a TLA-100 rotor in a Beckman TL-100 table top ultracentrifuge at 51,000 rpm (89,000×g) for 30 minutes at 4° C. After centrifugation the tubes are placed on ice. Whey is collected from the chilled tubes using an 18 gauge needle. Care is taken to leave the casein pellet and the upper cream layer undisturbed in the tube. Any solids or cream that co-transfer during the initial recovery are removed from the initial whey fraction by centrifugation 12,000 rpm for 30 minutes at 4° C. in a TMA-4 rotor in a Tomy MTX-150 centrifuge. Thereafter, the whey-containing tubes are place on ice and the whey is again recovered using a fresh 18 gauge needle.

(2) Pigs

Similar methods are employed to prepare milk and whey from pigs, in accordance with standard practice for obtaining and working with milk from normal pigs.

### EXAMPLE 6

### ELISA Assay of Prothrombin in Milk and Whey of Transgenic Animals

ELISA assays are used to measure the amount of prothrombin in milk from transgenic animals. One ELISA uses a monoclonal antibody, 7D7B10, that specifically recognizes the amino terminal region of prothrombin. The other ELISA uses a polyclonal anti-human Prothrombin antiserum. Except for the difference in the recognition reagent, the ELISAs are essentially the same and are carried out using the procedure detailed below.

Microtiter plate wells are coated overnight at 4° C. with 3 μg/ml of the monoclonal antibody in 50 μl of 0.1 M sodium bicarbonate buffer, pH 8.3. Afterward the wells are washed once with TET buffer (0.01 M Tris pH 7.5; 0.01 M EDTA; 0.02%; Tween-20, pH 7,45). They then are blocked with 400 μl per well of 1% BSA in PBS for 1 hour at 37° C. Thereafter the wells are washed with TET buffer five times. 100 μl of sample or control whey is introduced into the wells. The control whey is from normal animals and is spiked with various amounts of a reference prothrombin preparation to provide a calibration curve for the ELISA results. The whey samples are incubated in the wells to allow prothrombin therein to bind to the immobilized prothrombin-specific antibodies. The wells then are washed five times with TET buffer. Horse radish peroxidase (HRP) conjugated to rabbit anti-prothrombin is diluted 1:1,000 in 0.1% BSA/TET, 100 μl of the diluted HRP conjugate is added to each well and incubated for 2 hours at room temperature while shaking at 100 rpm. After the incubation, the conjugate-containing solution is removed from the wells. The wells are washed 5 times with TET buffer. Then 100 μl of a stock solution of orthophenyldiamine (OPD) is added to each well. (The stock solution is made by dissolving one tablet of OPD in 20 ml of 0.1 M citrate-phosphate buffer (pH 5.0). The OPD solution is incubated in the wells for 10 minutes at room temperature and then the reaction is stopped by adding 1 N sulfuric acid. The extent of the reaction is determined by measuring optical absorption of the acidified OPD solution in each well at 490 nm.

Fiduciary curves are developed for the ELISA assay for both the monoclonal and the polyclonal reagents using a standard preparation of human prothrombin. The concentration of prothrombin in the whey samples and in the milk from which they were derived is interpolated from the fiduciary curves. Milk and whey from normal non-transgenic mice or mice transgenic for other proteins obtained and treated the same as milk from the test animals is used for negative controls.

Results obtained by the two ELISAs are in close agreement. Almost all of the animals that are shown by PCR to be transgenic for the WAP-huPT provide significant levels of prothrombin in their milk, generally between 0.5 to 5.0 mg/ml.

Milk and whey from pigs transgenic for human prothrombin is tested in much the same way as described above for mice. The results from assays of transgenic pig milk shows prothrombin in the milk at levels between 0.5 and 5.0 mg/ml.

### EXAMPLE 7

### GLA in Prothrombin Produced in Transgenic Animals

Standard ELISAs are performed to assay prothrombin in whey from milk of transgenic animals. Normal human prothrombin is spiked into normal mouse milk whey at varying concentrations and assayed by the same protocol. Finally, prothrombin without Gla regions is assayed by the ELISA assay.

All three types of samples are loaded in 25 mM EDTA onto immunoaffinity columns specific for GLA. Unbound material is washed away and the columns then are treated with several washes of 25 mM CaCl₂. Material eluting in each wash is collected and assayed by the prothrombin ELISA. GLA-less prothrombin remained bound to the column in the presence of CaCl₂. Standard GLA elutes in the presence of CaCl₂. Prothrombin in whey from transgenic animals behaves like the normal prothrombin. The results indicate that the transgenic prothrombin is γ-carboxylated like the native molecule.

### EXAMPLE 8

### Chromogenic Assay of Thrombotic Amidolytic Activity

20 μL of sample or control is mixed with 80 μL of 1 μg/ml snake venom activator 20 mM Tris HCl, 150 mM NaCl, 0.2% NaAzide, pH 7.4 (“ctivation buffer”). The mixture is incubated at 37° C. for one hour. 100 μL of 0.25 mM thrombin chromogenic substrate is then added and the color is allowed to proceed for 1 to 3 hours. Absorbance at 450 nm is determined and the activity of each sample is determined by interpolation using a reference curve based on human plasma prothrombin diluted in activation buffer. Regarding amidolytic activity assays of this type see, for instance, U.S. Pat. No. 5,476,777 to Holly et al. for Methods for producing thrombin, column 28, lines 33 et seq. in particular, which is incorporated herein by reference in its entirety in parts pertinent to the foregoing description of thrombotic amidolytic activity assay.

### EXAMPLE 9

### Prothrombin in Milk of Transgenic Mice from Embryos Injected with WAP6PT1

(1) Construction of WAP6PT1

WAP6PT1 (also referred to as WAP6PT) was constructed essentially as described in Example 1 for WAP-huPTC-01. The two constructs are essentially the same except for minor differences that facilitate manipulation of the prothrombin expression cassette.

(2) Preparation of WAP6PT for Injection

pUCWAP6PT was digested with the restriction enzyme Not to release the DNA construct (WAP6PT) from the vector (pUCNotI+). The reaction mixture containing both

DNA fragments was placed on a 0.8% agarose gel and subjected to gel electrophoresis to separate the WAP6PT insert from other fragments. The band corresponding to the WAP6PT construct was cut out and subjected to Agarase treatment. Following Agarase treatment the reaction mixture was layered onto a NaCl step gradient (5% to 25% in 2.5% intervals) and centrifuged at 25,000 rpm for 6 hrs at 25° C., as described by Chin-Tih, Biotechniques 10(4): 446-450 (April 1991). 0.5 ml fractions were collected from each tube following centrifugation. 10 μl of each fraction was sample was subjected to 1.0% gel electrophoresis to identify those that contained WAP6PT DNA. Fractions containing the construct were pooled and then dialyzed for 45 minutes in ultrapure water. DNA was precipitated from the dialyzed sample with NaClO₄ and isopropanol, and collected by centrifugation. The pellet containing the DNA was resuspended in injection buffer. The concentration of the DNA was adjusted to 3-5 μg/ml in injection buffer for microinjection.

(3) Embryo Injection

Mouse embryos were obtained and injected as described above in Example 3. Injected embryos were implanted as described above in Example 4.

(4) Detection of Transgenic WAP6PT DNA

The presence of WAP6PT DNA in mice from injected embryos was detected by PCR using WAP6PT-specific primers, much as described above in Example 4. In brief, tails were snipped from mice after weaning. Tail DNA was isolated by phenol-chloroform extraction. The DNA was tested for incorporation of WAP6PT by PCT using primers specific to the mouse WAP promoter region and designed so that only DNA from animals incorporating multiple successive copies of the transgene yields a PCR product. The PCR reactions were electrophoresed in agarose containing 5 μg/ml ethidium bromide and the products were visualized under UV light.

(5) Breeding of Transgenic Mice

Mice that were determined to be transgenic by PCR were bred and allowed to complete gestation.

(6) Prothrombin in Milk and Whey of Transgenic Mice

Milk samples were obtained from transgenic females mice at early (days 5-7), mid (days 10-12), and late (days 15-16) stages of lactation, using the methods described above in Example 5. The volume of milk obtained was measured and a sample was removed and diluted 1:2 in prothrombin dilution buffer (40 mM Tris/200 mM EDTA/200 mM NaCl, pH 7.4). When possible, a sample of undiluted whole milk also was immediately frozen at −70° C. The diluted samples were centrifuged at 14,000 rpm in a microcentrifuge for 30 minutes at 4° C. and then chilled for between 10 and 30 minutes at 4° C. Samples then were removed from the cold room. The clarified whey was removed from the samples and placed in lean tubes. The fat and precipitate layers were removed from each sample and discarded.

(7) Western Blot Analysis of Prothrombin in Whey Samples

Whey samples were further analyzed by western blotting for the presence of transgenic prothrombin. 5 μl of each whey sample was diluted to 100 μl in SDS-PAGE Tris/glycine reducing cocktail, which provides e1 μl of whey in each 20 μls loaded on the gel from this solution. A human prothrombin standard (Enzyme Research Laboratories, South Bend, Ind.) was diluted in reducing cocktail to 100 ngs per 20-μl and run on the gel as a reference standard alongside the whey samples. As an additional reference, whey from control non-transgenic animals, prepared and diluted identically to the transgenic samples, also was run on the gel. For quantitative estimates of expression levels, multiple dilutions of both the transgenic whey sample and the human reference material were prepared and loaded to the same gel. The references and the whey samples were loaded onto a pre-cast 7.5% polyacrylamide gels (Bio-Rad, Hercules, Calif.) and electrophoresed at 200V until the dye front was at or just eluting off the bottom of the gel.

The separated proteins were transferred from the gels onto PVDF membranes (Bio-Rad) using the Novex X-Cell II™ Blot module (Invitrogen, Carlsbad, Calif.) according to manufacturer's recommendations, except with an 18 to 24 hour transfer time. After transfer, membranes were blocked in TBST-Casein (25 mM Tris, pH 7.2/50 mM NaCl/0.05% Tween 20/0.5% Casein) for 1-3 hours at 37° C. Sheep anti-human prothrombin antibody (ERL) was added to the blocking buffer at a 1:1000 dilution and membranes were allowed to incubate for at least one additional hour. The primary antibody solution was decanted, and membranes were extensively washed in deionized water before being place in a fresh aliquot of blocking buffer. Donkey anti-sheep antibody conjugated to horseradish peroxidase (Sigma, St. Louis, Mo., product number 3415) was added at 1:1000 dilution. After an additional 30-75 minutes at 37° C., the membranes were again extensively water washed before color development using the metal-enhanced DAB kit (Pierce, Rockford, Ill.). Blots were thoroughly dried on filter paper before further handling. After the western blot was completed as described above, the membrane was scanned using a Shimadzu CS-9000 dual-wavelength, flying spot densitometer (350 nm, 0.4×5 mm beam size).

### (8) Transgenic Mice from Embryos Injected with WAP6PT at 5 μg/ml

2,282 embryos were colleted, of which 545 were rejected because they appeared to be unfertilized. 1,737 of the embryos were injected with WAP6PT DNA at 5 μg/ml. 986 of the injected embryos were transferred into 27 recipient females (36.5 zygotes per recipient on average). 12 pregnancies resulted (44% pregnancy rate) and produced 34 pups (average of 2.8 pups per liter). 3 of these 34 pups were transgenic for WAP6PT by PCR (8.8% of the total). Copy numbers of the WAP6PT construct in the transgenic mice is estimated by Southern analysis.

(9) Transgenic Mice from Embryos Injected with WAP6PT DNA at 3 μg/ml

2,782 embryos were colleted, of which 640 were rejected because they appeared to be unfertilized. 2,021 of the embryos were injected with WAP6PT DNA at 3 μg/ml. 1,207 of the injected embryos were transferred into 29 recipient females (40.6 zygotes per recipient on average). 19 pregnancies resulted (65% pregnancy rate) and produced 99 pups (average of 5.2 pups per liter). WAP6PT DNA was found by PCR in 19 out of 87 of pups that were screened for the transgene (22%). Copy numbers of the WAP6PT construct in the transgenic mice is estimated by Southern analysis.

(10) Prothrombin in Milk of WAP6PT Transgenic Mice

28 samples of milk from 9 different transgenic mice were analyzed for the presence of transgenic prothrombin. Prothrombin was detected in 13 samples from 5 different mice. 9 of the samples were from early lactations. Very high levels of prothrombin were detected in milk from one mouse (No. 54) throughout lactation. Analysis of samples from second lactations indicates that individual expression patterns of the mice are consistent over multiple lactations.

Western analysis showed appropriate bands for prothrombin in samples from transgenic mice with both high and low prothrombin milk concentrations.

The prothrombin concentration in one high expressing mice was estimated quantitatively. A sample from early lactation was diluted to 0.01 μl whey/1 l gel-loading sample. 20, 10, and 5 μl of this sample were loaded to a gel alongside samples containing 400, 200, 100. or 50 ngs of human reference (each including 1 μl of non-transgenic whey). After electrophoresis the gel was blotted onto a filter, probed and developed using standard methods as described above. The densities of the resulting bands in the filter then were determined. The amount of prothrombin in each sample was derived from the densities by interpolation from a standard curve based on dilutions of a reference standard preparation as described above. The early lactation milk from this mouse contained 5.5-±2.1 mg/ml of prothrombin, based on the quantitative Western analysis.

(11) Germ Line Transmission of WAP6PT1 DNA

22 mice transgenic for the WAP6PT1 DNA construct have been bred and each transgenic line is being evaluated for transmission of the WAP6PT1 DNA and for prothrombin expression in milk, as noted in part above. Of the three transgenic mice produced from embryos injected with 5 μg/ml WAP6PT1 DNA the one female transmitted the transgene to offspring and the two males did not. Transmission by the transgenic mice produced from embryos injected with 3 μg/ml WAP6PT1 DNA is evaluated in the same way.

1-55. (canceled)

56. A non-human transgenic livestock mammal expressing a recombinant human prothrombin protein into a milk composition, wherein said expressed protein has a concentration of at least 1.5 mg/ml.

57. The transgenic livestock mammal of claim 56, wherein the mammal is a pig, a sheep, a goat, a cow or a horse.

58. The transgenic livestock mammal of claim 56, wherein said milk composition accumulates in a mammary gland.

59. The transgenic livestock mammal of claim 56, wherein said mammal further comprises a promoter operatively linked to a nucleic acid encoding said recombinant prothrombin.

60. The transgenic livestock mammal of claim 59, wherein said promoter is derived from a gene selected from the group consisting of whey acidic protein genes, casein genes, lactalbumin genes, and beta lactoglobulin genes.

61. The transgenic livestock mammal of claim 60, wherein said whey acidic protein gene comprises a whey acidic protein promoter selected from the group consisting of a mouse long whey acidic protein promoter and a pig whey acidic protein promoter.

62. The transgenic livestock mammal of claim 56, wherein said recombinant prothrombin comprises a post-translational modification selected from the group consisting of the group consisting of glycosylation, γ-carboxylation and proteolytic processing.

63. The transgenic livestock mammal of claim 62, wherein said γ-carboxylation of said recombinant prothrombin comprises at least ten glutamic acid residues.

64. The transgenic livestock mammal of claim 62, wherein said glycosylation of said recombinant prothrombin comprises at least three asparagine residues.

65. The transgenic livestock mammal of claim 62, wherein said proteolytic processing of said recombinant prothrombin comprises enzymatic cleavage.

66. The transgenic livestock mammal of claim 65, wherein said enzymatic cleavage comprises an enzyme selected from the group consisting of Factor Xa, Factor Va, thrombin, and combinations thereof.

67. The transgenic livestock mammal of claim 65, wherein said enzymatic cleavage further comprises calcium.

68. The transgenic livestock mammal of claim 65, wherein said enzymatic cleavage further comprises phospholipids.

69. The transgenic livestock mammal of claim 62, wherein said proteolytic processing converts said recombinant prothrombin to recombinant thrombin.
